(19) United States
(12) Patent Application Publication
(10) Pub. No.: US 2023/0045644 A1
(43) Pub. Date: Feb. 9, 2023

(54) SIEVE BED ASSEMBLY WITH AN IDENTIFICATION DEVICE

(71) Applicant: ResMed Asia Pte. Ltd., Singapore (SG)

(72) Inventors: Devona Yinqiu Poon, Singapore (SG); Henry Rohlich, Singapore (SG); Sandhya Verma, Singapore (SG); Joo Poh Tan, Singapore (SG); Siew Ying Koh, Singapore (SG)

(21) Appl. No.: 17/790,072

(22) PCT Filed: Dec. 24, 2020
(86) PCT No.: PCT/IB2020/062456

§ 371 Date: Jun. 29, 2022

Related U.S. Application Data
(60) Provisional application No. 62/955,765, filed on Dec. 31, 2019.

(51) Int. Cl. A61M 16/10; B01D 53/04; B01D 53/047; G05B 15/02; G16H 40/20

(52) U.S. Cl. CPC A61M 16/101; B01D 53/0415; B01D 53/0476; G05B 15/02; G16H 40/20; A61M 2205/60; B01D 2256/12; B01D 2257/102; B01D 2259/4533; B01D 2259/4541; B01D 2253/108

(57) ABSTRACT

A sieve bed assembly monitoring system is disclosed. The system includes a sieve bed assembly including a canister having an intake; adsorbent material to produce oxygen enriched air from compressed air in a swing adsorption process; and an identification device including identification data for the sieve bed assembly, wherein the identification data is capable of uniquely identifying the sieve bed assembly. The system includes an oxygen concentrator having a retention mechanism to retain the sieve bed assembly, a compressor supplying compressed air to the intake of the canister, a controller, a transceiver and a reader operable to read the identification data from the identification device. The controller reads the identification data and transmits the read identification data via the transceiver. A remote external device receives the read identification data from transceiver.

### CROSS-REFERENCE TO RELATED APPLICATIONS

This application claims the benefit of, and priority to, U.S. Provisional Patent Application No. 62/955,765, filed on Dec. 31, 2019, which is hereby incorporated by reference herein in its entirety.

### TECHNICAL FIELD

The present disclosure relates generally to portable oxygen concentrators (POCs), and more specifically to a sieve bed assembly having an identification device for tracking of the use of the sieve bed assembly in a POC.

### BACKGROUND

There are many users that require supplemental oxygen as part of Long Term Oxygen Therapy (LTOT). Currently, the vast majority of users that are receiving LTOT are diagnosed under the general category of Chronic Obstructive Pulmonary Disease (COPD). This general diagnosis includes common diseases such as Chronic Bronchitis, Emphysema, and related pulmonary conditions. Other users may also require supplemental oxygen, for example, obese individuals to maintain elevated activity levels, users with cystic fibrosis or infants with broncho-pulmonary dysplasia.

Doctors may prescribe oxygen concentrators or portable tanks of medical oxygen for these users. Usually a specific continuous oxygen flow rate is prescribed (e.g., 1 litre per minute (LPM), 2 LPM, 3 LPM, etc.). Experts in this field have also recognized that exercise for these users provide long term benefits that slow the progression of the disease, improve quality of life and extend user longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and they are heavy, weighing about 50 pounds when mounted.

Oxygen concentrators have been in use for about 50 years to supply users suffering from respiratory insufficiency with supplemental oxygen via oxygen enriched gas. Traditional oxygen concentrators used to provide these flow rates have been bulky and heavy making ordinary ambulatory activities with them difficult and impractical. Recently, companies that manufacture large stationary home oxygen concentrators began developing portable oxygen concentrators (POCs). The advantage of POCs is that they can produce a theoretically endless supply of oxygen enriched gas. In order to make these devices small for mobility, the various systems necessary for the production of oxygen enriched air are condensed.

Oxygen concentrators may implement processes such as vacuum swing adsorption (VSA), pressure swing adsorption (PSA), or vacuum pressure swing adsorption (VPSA) to produce oxygen enriched air. For example, pressure swing adsorption (PSA) involves using a compressor to increase gas pressure inside a canister, known as a sieve bed, that contains particles of a gas separation adsorbent that attracts nitrogen more strongly than it does oxygen. Ambient air usually includes approximately 78% nitrogen and 21% oxygen with the balance comprised of argon, carbon dioxide, water vapor and other trace gases. If a feed gas mixture such as air, for example, is passed under pressure through a sieve bed, part or all of the nitrogen will be adsorbed by the sieve bed, and the gas coming out of the vessel will be enriched in oxygen. When the sieve bed reaches the end of its capacity to adsorb nitrogen, it can be regenerated by reducing the pressure, thereby releasing the adsorbed nitrogen. It is then ready for another “PSA cycle” of producing oxygen enriched gas such as oxygen enriched air. By alternating canisters in a two-canister system, one canister can be concentrating oxygen (the so-called “adsorption phase”) while the other canister is being purged (the “purge phase”). This alternation results in a continuous separation of the oxygen from the nitrogen. In this manner, oxygen can be continuously concentrated out of the air for a variety of uses which include providing supplemental oxygen to users. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Vacuum swing adsorption (VSA) provides an alternative gas separation technique. VSA typically draws the gas through the separation process of the sieve beds using a vacuum such as a compressor configured to create a vacuum with the sieve beds. Vacuum Pressure Swing Adsorption (VPSA) may be understood to be a hybrid system using a combined vacuum and pressurization technique. For example, a VPSA system may pressurize the sieve beds for the separation process and also apply a vacuum for purging of the beds.

The gas separation adsorbents used in POCs have a very high affinity for water. This affinity is so high that it overcomes nitrogen affinity, and thus when both water vapor and nitrogen are available in a feed gas stream (such as ambient air), the adsorbent will preferentially adsorb water vapor over nitrogen. Furthermore, when it is adsorbed, water does not desorb as easily as nitrogen. As a result, water molecules remain adsorbed even after regeneration and thus block the adsorption sites for nitrogen. Some reduction of this effect may be achieved by the addition of a desiccant material in the sieve bed. However, over time and use, the desiccant becomes saturated and water accumulates on the adsorbent, which becomes less and less efficient for nitrogen adsorption, to the point where the sieve bed needs to be replaced because no further oxygen concentration can take place. Such sieve beds may be referred to as exhausted or deactivated.

It is currently not possible to track individual produced sieve beds and their use. Thus, suppliers may often replace an entire batch of sieve beds as part of a maintenance schedule rather than replace an individual sieve bed. Such a practice wastes resources as certain sieve beds in a replaced batch may not be exhausted as each individual sieve bed may have different exhaustion rates based on the use of the POCs. Further, the sieve beds may vary between manufacturing batches and there is presently no way to determine whether there may be production issues based on the use of the sieve beds.

There is thus a need for a system that allows the determination of unique identification data for sieve beds installed in oxygen concentrators. There is another need to collect data from sieve beds to determine the quality of production in a manufacturing batch.

### SUMMARY

The present disclosure relates to a system for tracking the use of a sieve bed assembly in an oxygen concentrator. Unique identification data, in formats such as indicia, symbols, or code, is assigned to each sieve bed assembly so that the identification data is capable of uniquely identifying the sieve bed assembly. As such, the identification data is unique in that it is associated with a particular sieve bed assembly and no other sieve bed assembly. The indicia or code may comprise at least one number, at least one alphabetic character or a combination thereof. The unique identification data may be a unique serial number or a unique random number. For instance, when the unique identification data is a serial number, the serial number is recorded in a tag device such as an RFID tag on the sieve bed assembly. When the sieve bed assembly is installed in the oxygen concentrator, a reader such as an RFID reader on the oxygen concentrator can read the serial number from the tag device. Once the RFID reader collects identification data (or identification information) such as the unique serial number of the sieve bed assembly, the identification information is uploaded to a remote external device, such as a cloud server, wherein the cloud server comprises a database. As such, information can be collected and analyzed at a later point of time. For instance, information about the origin of the sieve bed assembly (e.g. the batch number of the sieve bed assembly) and the frequency of changing the sieve bed assembly can be tracked and correlated with the use of the sieve bed assembly in the oxygen concentrator.

One disclosed example is a sieve bed assembly monitoring system. The system has a sieve bed assembly that includes a canister having an intake; adsorbent material to produce oxygen enriched air from compressed air in a swing adsorption process; and an identification device including unique identification data identifying the sieve bed assembly. The system has an oxygen concentrator having a compressor supplying compressed air to the intake of the canister, a controller, a transceiver and a reader operable to read the identification data from the identification device. The controller is operable to read the identification data and transmit the read identification data via the transceiver. The system also has a remote external device operable to receive the read identification data from the transceiver.

In another implementation of the disclosed example sieve bed assembly monitoring system, the identification device is one of an RFID tag sending an RFID signal to the reader or an NFC tag sending an NFC signal to the reader. In another implementation, the oxygen concentrator further includes a retention mechanism to retain the sieve bed assembly. The controller determines whether the sieve bed assembly is properly installed in the retention mechanism based on the strength of the signal received by the reader. In another implementation, the remote external device is a portable computing device. In another implementation, the remote external device is a cloud based server. In another implementation, the example sieve bed assembly monitoring system includes a database having manufacturing information relating to the sieve bed assembly. The cloud based server correlates the read identification data to the manufacturing information relating to the sieve bed assembly. In another implementation, the example sieve bed assembly monitoring system includes a database having operating information relating to the oxygen concentrator. The controller is collects operating information relating to the oxygen concentrator and sends the collected operating information via the transceiver to the cloud based server. The cloud based server correlate the read identification data to the collected operating information. In another implementation, the remote external device predicts an end of life of the sieve bed assembly and correlate the predicted end of life with the read identification data. In another implementation, the identification device is a label including a graphic of the identification data, and the reader is an optical scanner. In another implementation, the identification device is a readable and writable memory device.

Another disclosed example is a sieve bed assembly including a canister having an intake and adsorbent material for producing oxygen enriched air from compressed air in a swing adsorption process, wherein the adsorbent material is within the canister. The assembly further includes an identification device including identification data for the sieve bed assembly. The identification data is capable of uniquely identifying the sieve bed assembly. The identification device is configured to allow a reader to read the identification data from the identification device.

In another implementation of the disclosed example sieve bed assembly, the identification device is one of an RFID tag sending an RFID signal to the reader or an NFC tag sending an NFC signal to the reader. In another implementation, the identification device is a memory device. In another implementation, the memory device is an EEPROM.

Another disclosed example is a method of tracing a sieve bed assembly for installation in an oxygen concentrator. An identification device including identification data relating to a sieve bed assembly is attached to the sieve bed assembly. The identification data uniquely identifies the sieve bed assembly. The identification data is read from the identification device via a reader. The read identification data is stored in a database.

In another implementation of the disclosed example method, the reading is performed at a manufacturing facility. In another implementation, the reading is performed by a reader on an oxygen concentrator. In another implementation, the method includes sending the read identification data to a server via a transceiver on the oxygen concentrator. In another implementation, the method includes correlating the read identification data to manufacturing information relating to the sieve bed assembly. In another implementation, the method includes collecting operational data from the oxygen concentrator; transmitting the collected operational data to a database; and correlating the read identification data to the collected operating information. In another implementation, the identification device is one of an RFID tag sending an RFID signal to the reader or an NFC tag sending an NFC signal to the reader. In another implementation, the identification device is a label including a graphic of the identification data, and wherein the reader is an optical scanner. In another implementation, the method includes predicting an end of life of the sieve bed assembly; and correlating the predicted end of life with the read identification data. In another implementation, the sieve bed assembly includes a readable and writable memory device storing the identification data.

Another disclosed example is an oxygen concentrator that has a retention mechanism for holding a sieve bed assembly having a canister with an adsorbent material and an identification device including identification data uniquely identifying the sieve bed assembly. The oxygen concentrator includes a compression system including a compressor coupled to the canister. The compressor compresses air for the canister to produce oxygen enriched air in a swing adsorption process. The oxygen concentrator includes a reader operable to read the identification data from the identification device and a transceiver. A controller is coupled to the reader and the transceiver. The controller causes the reader to read the identification data and sends the read identification data to a remote external device via the transceiver.

In another implementation of the disclosed oxygen concentrator, the identification device is one of an RFID tag sending an RFID signal to the reader or an NFC tag sending an NFC signal to the reader. In another implementation, the oxygen concentrator includes a printed circuit board including the controller and the reader in fixed relation to the retention mechanism. The controller determines whether the sieve bed assembly is properly installed in the retention mechanism based on the strength of the signal received by the reader. In another implementation, the remote external device is a cloud based server. In another implementation, the remote external device is a portable computing device. In another implementation, the remote external device is operable to predict an end of life of the sieve bed assembly and correlate the predicted end of life with the read identification data. In another implementation, the identification device is a readable and writable memory device.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1A depicts an oxygen concentrator in accordance with one form of the present technology;

FIG. 1B is a schematic diagram of the components of the oxygen concentrator of FIG. 1A;

FIG. 1C is a side view of the main components of the oxygen concentrator of FIG. 1A;

FIG. 1D is a perspective side view of a compression system of the oxygen concentrator of FIG. 1A;

FIG. 1E is a side view of a compression system that includes a heat exchange conduit;

FIG. 1F is a schematic diagram of example outlet components of the oxygen concentrator of FIG. 1A;

FIG. 1G depicts an outlet conduit for the oxygen concentrator of FIG. 1A;

FIG. 1H depicts an alternate outlet conduit for the oxygen concentrator of FIG. 1A;

FIG. 1I is a perspective view of a disassembled canister system for the oxygen concentrator of FIG. 1A;

FIG. 1J is an end view of the canister system of FIG. 1I;

FIG. 1K is an assembled view of the canister system end depicted in FIG. 1J;

FIG. 1L is a view of an opposing end of the canister system of FIG. 1I to that depicted in FIGS. 1J and 1K;

FIG. 1M is an assembled view of the canister system end depicted in FIG. 1L;

FIG. 1N is a block diagram of a communication arrangement of example devices that may be in communication with the oxygen concentrator of FIG. 1A;

FIG. 1O depicts an example control panel for the oxygen concentrator of FIG. 1A;

FIG. 2A is a perspective view of an example sieve bed assembly that includes an identification device on a top surface of the sieve bed assembly;

FIG. 2B is a top view of the example sieve bed assembly of FIG. 2A;

FIG. 2C is a bottom view of the example sieve bed assembly of FIG. 2A;

FIG. 3 is a block diagram of the system to read the identification information from the installed sieve bed assembly by a POC using the sieve bed assembly in FIGS. 2A-2C; and

FIG. 4 is a block diagram of a POC component monitoring system that allows sieve bed assemblies to be tracked for POCs.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a portable oxygen concentrator system that uses a sieve bed assembly having an identification tag device (or tag device or identification device), such as a radio frequency identification (RFID) tag or a Near Field communication (NFC) tag, which encodes a unique identification data associated with the sieve bed assembly. Consequently, the identification data is capable of uniquely identifying the sieve bed assembly. The unique identification data may be transmitted in the form of a signal or be visually read in the form of indicia, symbols or code. The identification data may comprise at least one number, at least one alphabetic character, or a combination thereof. The unique identification data may be a unique serial number or a unique random number. The identification device including identification data for the sieve bed assembly may be on an exterior surface of the sieve bed assembly. The identification device may be located near a reader when the sieve bed assembly is attached to the oxygen concentrator. The reader may be mounted on a printed circuit board assembly (PCBA) for the oxygen concentrator or may be a stand-alone reader. The reader is configured such that it can read the tag device on the sieve bed assembly thereby obtaining the unique identification data. In other words, the reader is operable to read the identification data from the identification device. The controller of the oxygen concentrator may then transmit the identification data to a remote external device such as a cloud based server (or server). In other words, the controller is operable to read the identification data and transmit the read identification data via a transceiver to a remote external device such as a cloud based server. The remote external device is operable to receive the read identification data from the transceiver. The cloud based server (or server) may be a remote server. The automatic collection of the identification data of the sieve bed assembly allows the identification to be correlated with operational data from the oxygen concentrator as well as manufacturing data.

The system can optionally detect whether the sieve bed assembly is properly installed. This is because the controller is operable to determine whether the sieve bed assembly is properly installed based on the strength of the signal received by the reader. For instance, when a tag device such as an RFID tag and a reader such as an RFID reader are used, the quality of the RF signal from the RFID tag can be an indicator that the sieve bed assembly is properly installed. The quality of the RF signal may be correlated to the strength of the RF signal received by the RFID reader. As such, the strength of the RF signal received by the RFID reader may allow the system to detect whether the sieve bed assembly is properly installed. When the tag device is a NFC tag and the reader is an NFC reader, the quality of the NFC signal from the NFC reader can be correlated to the strength of the NFC signal received by the NFC reader. As such, the strength of the NFC signal received by the NFC reader may allow the system to detect whether the sieve bed assembly is properly installed.

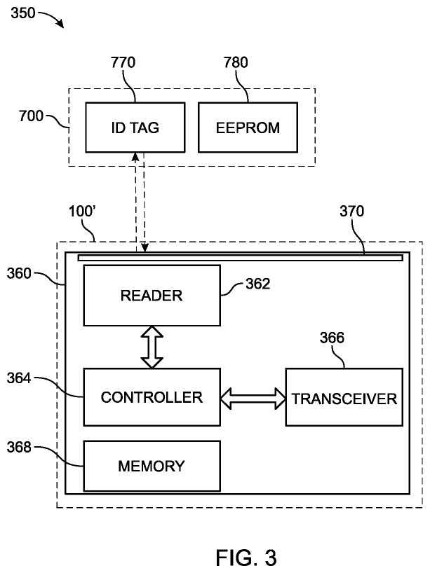

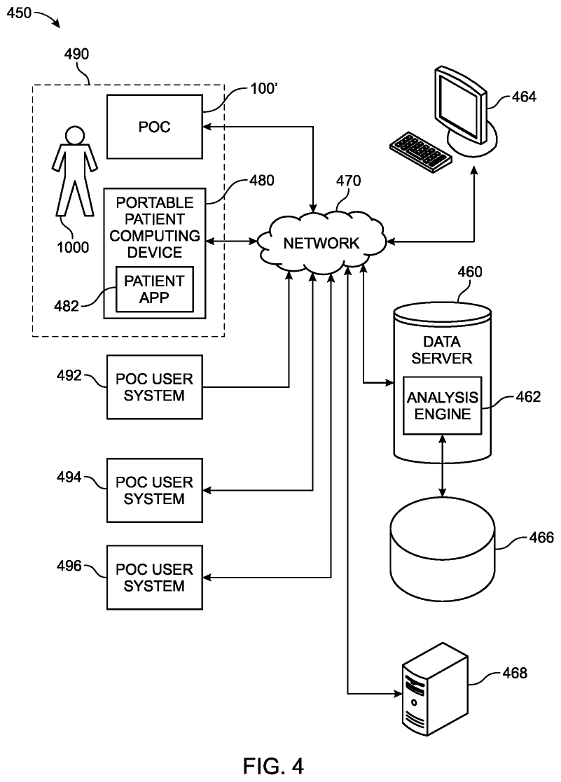

An example oxygen concentrator of the present technology may be considered in relation to the examples of the figures. The examples of the present technology may be implemented with any of the following structures and operations. It would be understood that various structures and operations of the oxygen concentrator 100 may also be found in an oxygen concentrator 100′ shown in FIGS. 3-4.

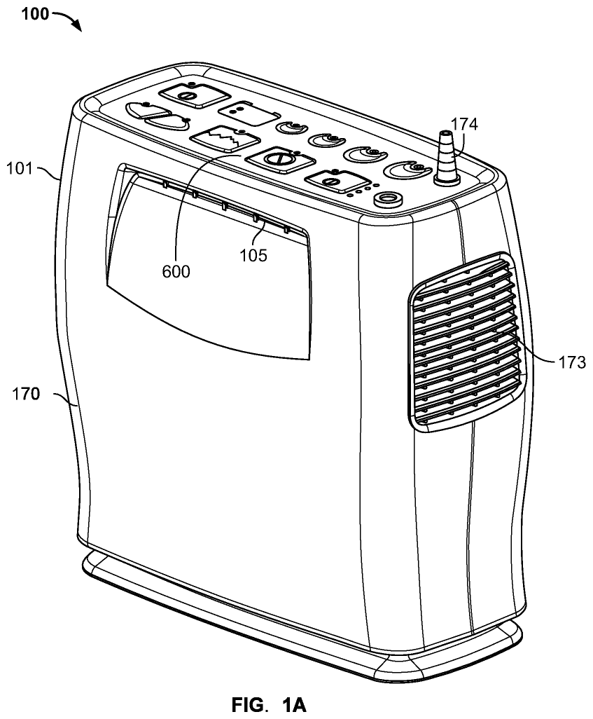

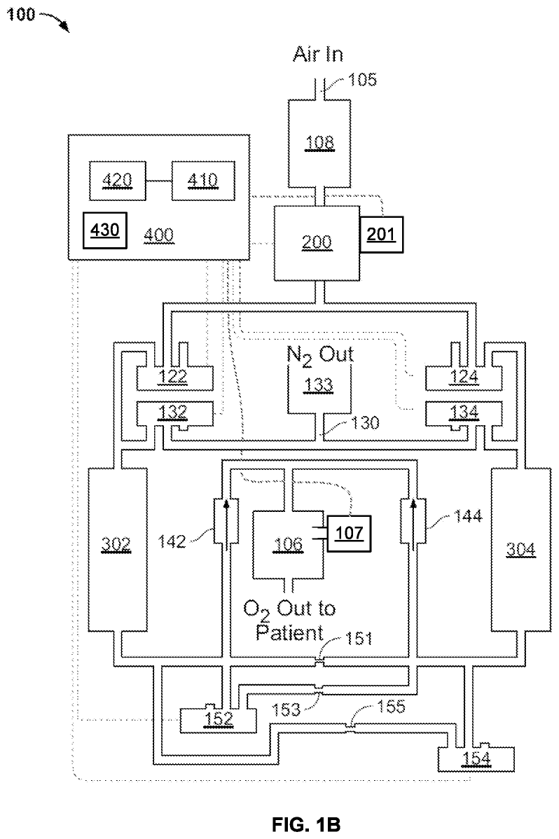

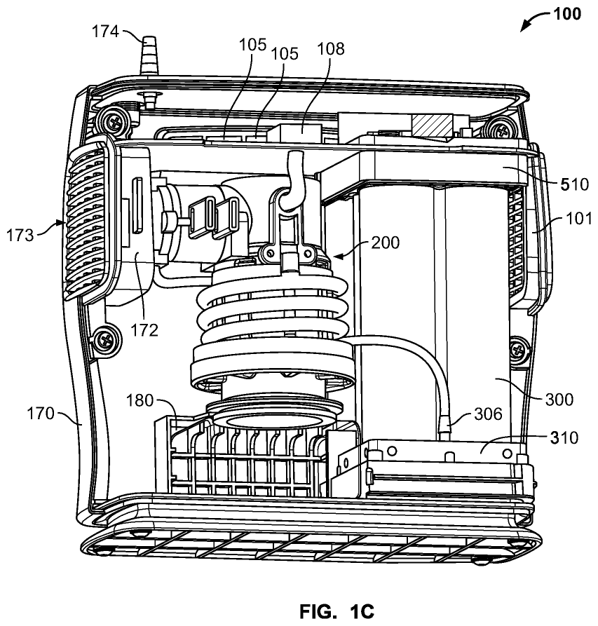

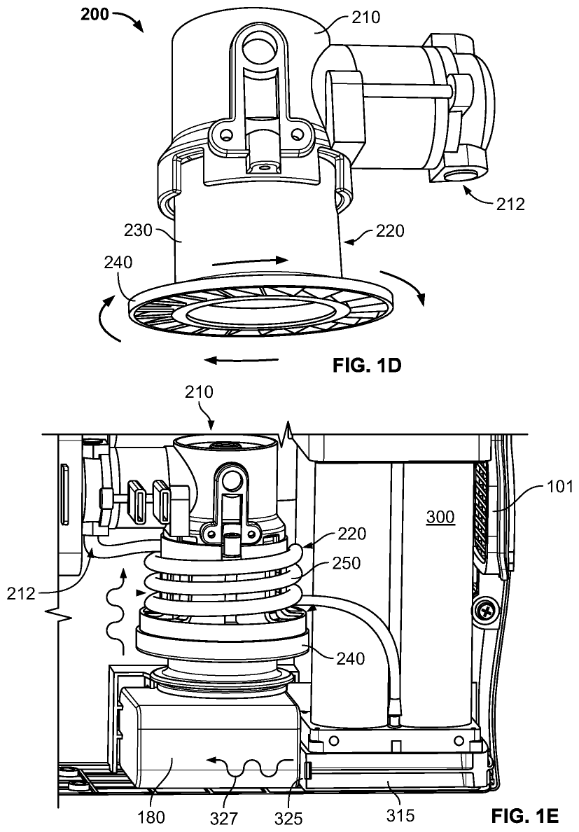

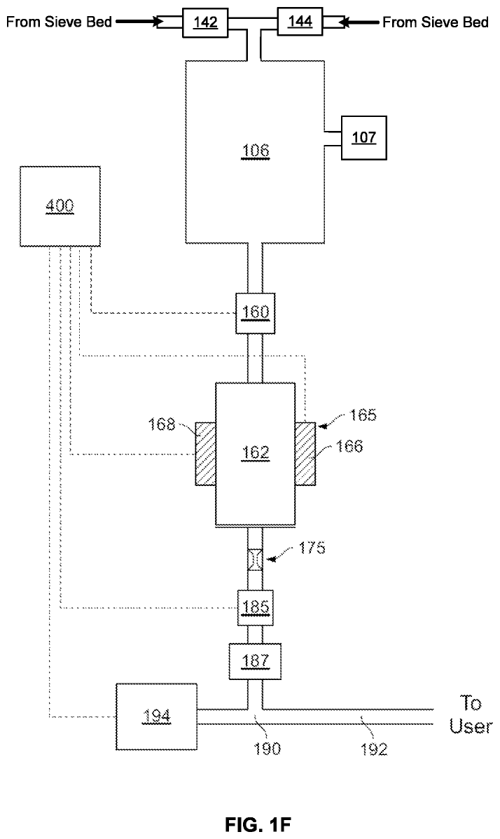

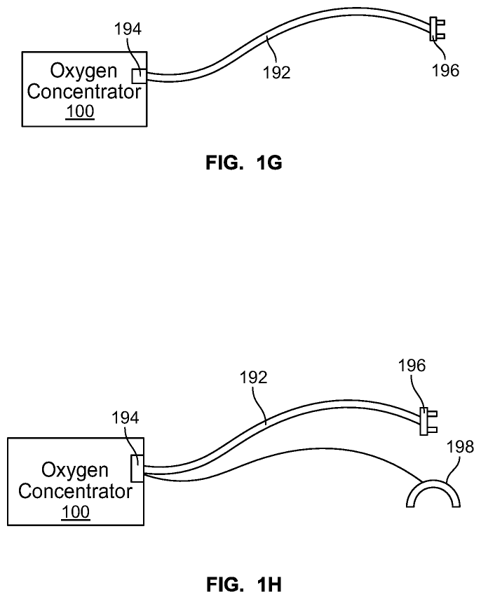

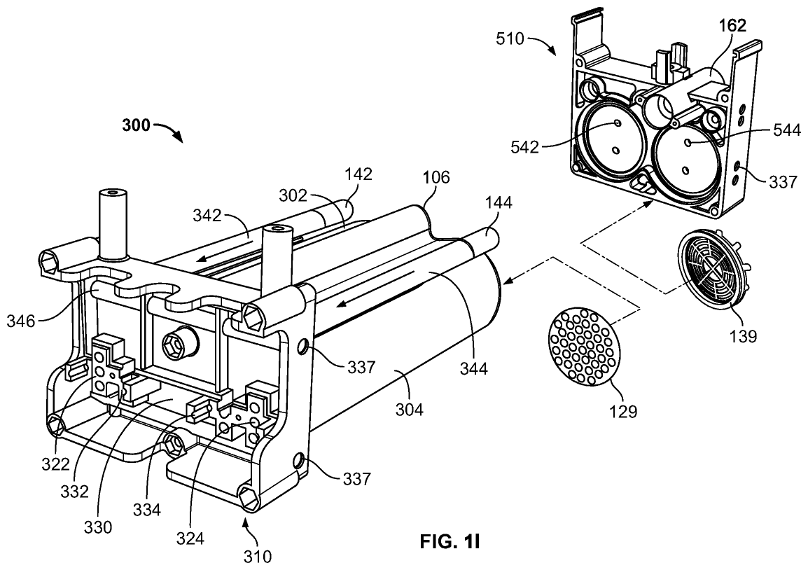

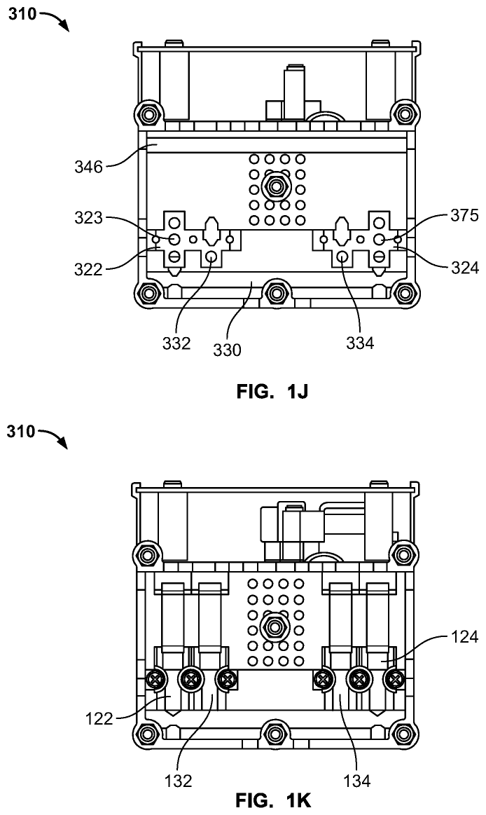

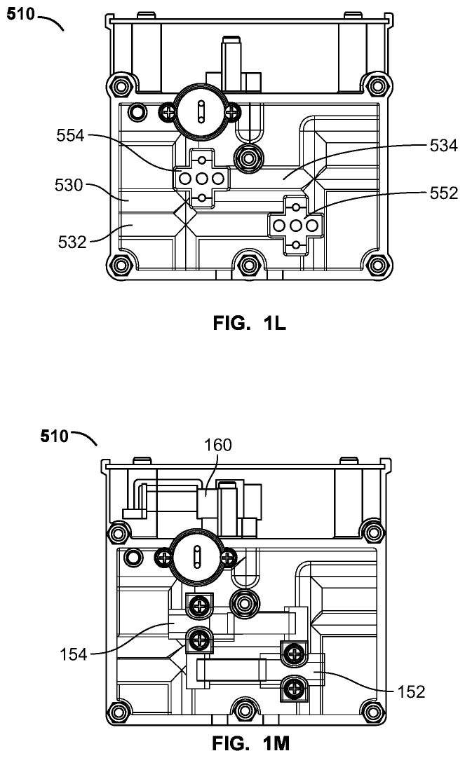

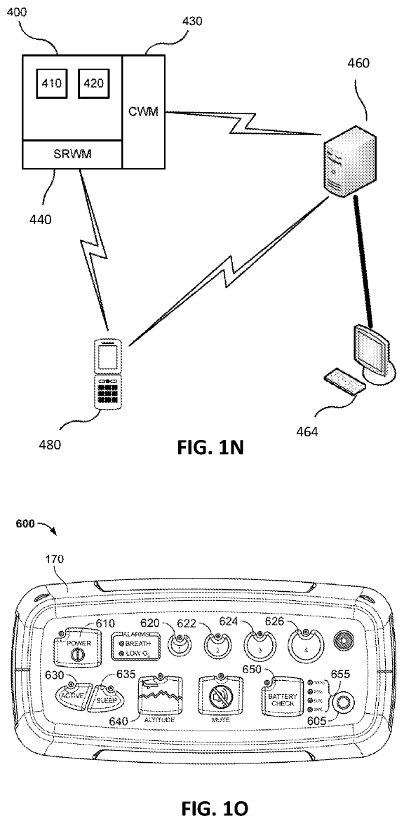

FIGS. 1A to 1O illustrate an implementation of an oxygen concentrator 100. Oxygen concentrator 100 may concentrate oxygen within an air stream to provide oxygen enriched air to a user. Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 pounds, less than about 15 pounds, less than about 10 pounds, or less than about 5 pounds. In an implementation, oxygen concentrator 100 has a volume of less than about 1000 cubic inches, less than about 750 cubic inches, less than about 500 cubic inches, less than about 250 cubic inches, or less than about 200 cubic inches.

As described herein, oxygen concentrator 100 uses a pressure swing adsorption (PSA) process (which is cyclic) to produce oxygen enriched air. However, in other implementations, oxygen concentrator 100 may be modified such that it uses a cyclic vacuum swing adsorption (VSA) process or a cyclic vacuum pressure swing adsorption (VPSA) process to produce oxygen enriched air.

FIG. 1A depicts an implementation of an outer housing 170 of an oxygen concentrator 100. In some implementations, outer housing 170 may be comprised of a light-weight plastic. The outer housing 170 includes compression system inlets 105, cooling system passive inlet 101 and outlet 173 at each end of the outer housing 170, outlet port 174, and control panel 600. Inlet 101 and outlet 173 allow cooling air to enter the housing, flow through the housing, and exit the interior of housing 170 to aid in cooling of the oxygen concentrator 100. The compression system inlets 105 allow air to enter the compression system. The outlet port 174 is used to attach a conduit to provide oxygen enriched air produced by the oxygen concentrator 100 to a user.

FIG. 1B illustrates a schematic diagram of a pneumatic system of an oxygen concentrator such as the oxygen concentrator 100 according to an implementation. The pneumatic system may concentrate oxygen within an air stream to provide oxygen enriched air to an outlet system (described below).

Oxygen enriched air may be produced from ambient air by pressurizing ambient air in canisters 302 and 304, which contain a gas separation adsorbent and are therefore referred to as sieve beds. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, Ill.; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the pneumatic system through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may reduce moisture and sound. For example, a water adsorbent material (such as a polymer water adsorbent material or a zeolite material) may be used to both adsorb water, from the incoming air and to reduce the sound of the air passing into the air inlet 105.

Compression system 200 may include one or more compressors configured to compress air. Pressurized air, produced by compression system 200, may be fed into one or both of the canisters 302 and 304. In some implementations, the ambient air may be pressurized in the canisters to a target pressure approximately in a range of 13-20 pounds per square inch gauge (psig). Other target pressure values may also be used, depending on the type of gas separation adsorbent disposed in the canisters.

As shown in FIG. 1B, in a particular disclosed implementation, the oxygen concentrator 100 has at least two canisters 302 and 304. Coupled to each canister 302 and 304 are inlet valves 122 and 124 and outlet valves 132 and 134. As shown in FIG. 1B, the inlet valve 122 is coupled to the “feed end” of the canister 302 and the inlet valve 124 is coupled to the feed end of the canister 304. Outlet valve 132 is coupled to the canister 302 and the outlet valve 134 is coupled to canister 304. The inlet valves 122 and 124 are used to control the passage of air from the compression system 200 to the respective canisters. The outlet valves 132 and 134 are used to vent exhaust gas from the respective canisters 302 and 304 to atmosphere. In some implementations, the inlet valves 122 and 124 and the outlet valves 132 and 134 may be silicon plunger solenoid valves. Other types of valves, however, may be used. Plunger valves offer advantages over other kinds of valves by being quiet and having low slippage.

In some implementations, a two-step valve actuation voltage may be generated to control the inlet valves 122 and 124 and the outlet valves 132 and 134. For example, a high voltage (e.g., 24 V) may be applied to an inlet valve to open the inlet valve. The voltage may then be reduced (e.g., to 7 V) to keep the inlet valve open. Using less voltage to keep a valve open may use less power. This reduction in voltage minimizes heat build-up and power consumption to extend run time from the power supply 180 (described below). When the power is cut off to the valve, it closes by spring action. In some implementations, the voltage may be applied as a function of time that is not necessarily a stepped response (e.g., a curved downward voltage between an initial 24 V and a final 7 V).

In an implementation, a controller 400 is electrically coupled to valves 122, 124, 132, and 134. The controller 400 includes one or more processors 410 operable to execute program instructions stored in a memory 420. The program instructions configure the controller to perform various predefined methods that are used to operate the oxygen concentrator, such as the methods described in more detail herein. The program instructions may include program instructions for generating for operating the inlet valves 122 and 124 out of phase with each other, i.e., when one of the inlet valves 122 or 124 is opened, the other valve is closed. During pressurization of the canister 302, the outlet valve 132 is closed and the outlet valve 134 is opened. Similar to the inlet valves, the outlet valves 132 and 134 are operated out of phase with each other. In some implementations, the voltages and the durations of the voltages used to open the input and output valves may be controlled by controller 400. The controller 400 may also include a transceiver 430 that may communicate with external devices to transmit data collected by the processor 410 or receive instructions from an external device for the processor 410.

Check valves 142 and 144 are coupled to the “product ends” of canisters 302 and 304, respectively. Check valves 142 and 144 may be one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve,” as used herein, refers to a valve that allows flow of a fluid (gas or liquid) in one direction and inhibits back flow of the fluid. The term “fluid” may include a gas or a mixture of gases (such as air). Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, the canister 302 is pressurized by compressed air produced in compression system 200 and passed into the canister 302. During pressurization of the canister 302, the inlet valve 122 is open, the outlet valve 132 is closed, the inlet valve 124 is closed and outlet valve 134 is open. The outlet valve 134 is opened when outlet valve 132 is closed to allow substantially simultaneous venting of canister 304 to atmosphere while canister 302 is being pressurized. After some time, the pressure in the canister 302 is sufficient to open check valve 142. Oxygen enriched air produced in canister 302 passes through check valve 142 and, in one implementation, is collected in an accumulator 106.

After some further time, the gas separation adsorbent in the canister 302 becomes saturated with nitrogen and is unable to separate significant amounts of nitrogen from incoming air. This point is usually reached after a predetermined time of oxygen enriched air production. In the implementation described above, when the gas separation adsorbent in the canister 302 reaches this saturation point, the inflow of compressed air is stopped and the canister 302 is vented to remove nitrogen. During venting of the canister 302, the inlet valve 122 is closed, and the outlet valve 132 is opened. While the canister 302 is being vented, the canister 304 is pressurized to produce oxygen enriched air in the same manner described above. Pressurization of the canister 304 is achieved by closing the outlet valve 134 and opening the inlet valve 124. After some time, the oxygen enriched air exits the canister 304 through the check valve 144.

During venting of the canister 302, the outlet valve 132 is opened allowing exhaust gas (mainly nitrogen) to exit the canister 302 to atmosphere through the concentrator outlet 130. In an implementation, the vented exhaust gas may be directed through the muffler 133 to reduce the noise produced by releasing the pressurized gas from the canister. As exhaust gas is vented from the canister 302, the pressure in the canister 302 drops, allowing the nitrogen to become desorbed from the gas separation adsorbent. The desorption of the nitrogen resets the adsorbent in the canister 302 to a state that allows renewed separation of nitrogen from an air stream. The muffler 133 may include open cell foam (or another material) to muffle the sound of the gas leaving the oxygen concentrator. In some implementations, the combined muffling components/techniques for the input of air and the output of oxygen enriched air may provide for oxygen concentrator operation at a sound level below 50 decibels.

During venting of the canisters 302 and 304, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air.

In some implementations, nitrogen removal may be assisted using an oxygen enriched air stream that is introduced into the canister from the other canister or stored oxygen enriched air. In an exemplary implementation, a portion of the oxygen enriched air may be transferred from the canister 302 to the canister 304 when the canister 304 is being vented of exhaust gas. Transfer of oxygen enriched air from the canister 302 to the canister 304 during venting of the canister 304, helps to desorb nitrogen from the adsorbent by lowering the partial pressure of nitrogen adjacent the adsorbent. The flow of oxygen enriched air also helps to purge desorbed nitrogen (and other gases) from the canister. In an implementation, oxygen enriched air may travel through flow restrictors 151, 153, and 155 between the two canisters 302 and 304. The flow restrictor 151 may be a trickle flow restrictor. The flow restrictor 151, for example, may be a 0.009 D flow restrictor (e.g., the flow restrictor has a radius 0.009″ which is less than the diameter of the tube it is inside). Flow restrictors 153 and 155 may be 0.013 D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective conduits. In some implementations, the press fit flow restrictors may be made of sapphire, metal or plastic (other materials are also contemplated).

Flow of oxygen enriched air between the canisters is also controlled by use of a valve 152 and a valve 154. The valves 152 and 154 may be opened for a short duration during the venting process (and may be closed otherwise) to prevent excessive oxygen loss out of the purging canister. Other durations are also contemplated. In an exemplary implementation, the canister 302 is being vented and it is desirable to purge the canister 302 by passing a portion of the oxygen enriched air being produced in the canister 304 into the canister 302. A portion of oxygen enriched air, upon pressurization of the canister 304, will pass through a flow restrictor 151 into the canister 302 during venting of the canister 302. Additional oxygen enriched air is passed into the canister 302, from the canister 304, through the valve 154 and the flow restrictor 155. The valve 152 may remain closed during the transfer process, or may be opened if additional oxygen enriched air is needed. The selection of appropriate flow restrictors 151 and 155, coupled with controlled opening of the valve 154 allows a controlled amount of oxygen enriched air to be sent from the canister 304 to the canister 302. In an implementation, the controlled amount of oxygen enriched air is an amount sufficient to purge the canister 302 and minimize the loss of oxygen enriched air through venting the valve 132 of the canister 302. While this implementation describes venting of the canister 302, it should be understood that the same process can be used to vent the canister 304 using the flow restrictor 151, the valve 152 and the flow restrictor 153.

The pair of equalization/vent valves 152 and 154 work with the flow restrictors 153 and 155 to optimize the gas flow balance between the two canisters 302 and 304. This may allow for better flow control for purging one of the canisters 302 and 304 with oxygen enriched air from the other of the canisters. It may also provide better flow direction between the two canisters 302 and 304. It has been found that, while flow valves 152 and 154 may be operated as bi-directional valves, the flow rate through such valves varies depending on the direction of fluid flowing through the valve. For example, oxygen enriched air flowing from the canister 304 toward the canister 302 has a flow rate faster through the valve 152 than the flow rate of oxygen enriched air flowing from the canister 302 toward the canister 304 through the valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalizing the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, the oxygen concentrator 100 may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters 302 and 304 may lead to a negative pressure in the canisters 302 and 304. Valves (e.g., valves 122, 124, 132, and 134) leading to and from the canisters 302 and 304 are dynamically sealed rather than hermetically sealed. Thus, outside air may enter the canisters 302 and 304 after shutdown to accommodate the pressure differential. When outside air enters the canisters 302 and 304, moisture from the outside air may be adsorbed by the gas separation adsorbent. Adsorption of water inside the canisters 302 and 304 may lead to gradual degradation of the gas separation adsorbents, steadily reducing ability of the gas separation adsorbents to produce oxygen enriched air.

In an implementation, outside air may be inhibited from entering the canisters 302 and 304 after the oxygen concentrator 100 is shutdown by pressurizing both canisters 302 and 304 prior to shut down. By storing the canisters 302 and 304 under a positive pressure, the valves may be forced into a hermetically closed position by the internal pressure of the air in the canisters 302 and 304. In an implementation, the pressure in the canisters, 302 and 304 at shutdown, should be at least greater than ambient pressure. As used herein the term “ambient pressure” refers to the pressure of the surroundings in which the oxygen concentrator 100 is located (e.g. the pressure inside a room, outside, in a plane, etc.). In an implementation, the pressure in the canisters 302 and 304, at shutdown, is at least greater than standard atmospheric pressure (i.e., greater than 760 mmHg (Torr), 1 atm, 101,325 Pa). In an implementation, the pressure in the canisters 302 and 304, at shutdown, is at least about 1.1 times greater than ambient pressure; is at least about 1.5 times greater than ambient pressure; or is at least about 2 times greater than ambient pressure.

In an implementation, pressurization of the canisters 302 and 304 may be achieved by directing pressurized air into each canister 302 and 304 from the compression system and closing all valves to trap the pressurized air in the canisters. In an exemplary implementation, when a shutdown sequence is initiated, inlet valves 122 and 124 are opened and outlet valves 132 and 134 are closed. Because the inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator 100 operates in an alternating pressurize/venting mode, at least one of the canisters 302 and 304 should be in a pressurized state at any given time. In an alternate implementation, the pressure may be increased in each canister 302 and 304 by operation of compression system 200. When the inlet valves 122 and 124 are opened, pressure between the canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

Referring to FIG. 1C, an implementation of an oxygen concentrator 100 is depicted. The oxygen concentrator 100 includes a compression system 200, a canister system 300, and a power supply 180 disposed within an outer housing 170. Inlets 101 are located in outer housing 170 to allow air from the environment to enter oxygen concentrator 100. The inlets 101 may allow air to flow into the compartment to assist with cooling of the components in the compartment. A power supply 180 provides a source of power for the oxygen concentrator 100. The compression system 200 draws air in through the inlet 105 and a muffler 108. The muffler 108 may reduce noise of air being drawn in by the compression system and also may include a desiccant material to remove water from the incoming air. The oxygen concentrator 100 may further include a fan 172 used to vent air and other gases from the oxygen concentrator via the outlet 173.

In some implementations, the compression system 200 includes one or more compressors. In another implementation, the compression system 200 includes a single compressor, coupled to all of the canisters of the canister system 300. Turning to FIGS. 1D and 1E, a compression system 200 is depicted that includes a compressor 210 and a motor 220. The motor 220 is coupled to the compressor 210 and provides an operating force to the compressor 210 to operate the compression mechanism. For example, the motor 220 may be a motor providing a rotatable component that causes cyclical motion of a component of the compressor 210 that compresses air. When the compressor 210 is a piston type compressor, the motor 220 provides an operating force which causes the piston of the compressor 210 to be reciprocated. Reciprocation of the piston causes compressed air to be produced by the compressor 210. The pressure of the compressed air is, in part, estimated by the speed that the compressor is operated at, (e.g., how fast the piston is reciprocated). The motor 220, therefore, may be a variable speed motor that is operable at various speeds to dynamically control the pressure of air produced by the compressor 210.

In one implementation, the compressor 210 includes a single head wobble type compressor having a piston. Other types of compressors may be used such as diaphragm compressors and other types of piston compressors. The motor 220 may be a DC or AC motor and provides the operating power to the compressing component of the compressor 210. The motor 220, in an implementation, may be a brushless DC motor. The motor 220 may be a variable speed motor configured to operate the compressing component of the compressor 210 at variable speeds. The motor 220 may be coupled to the controller 400, as depicted in FIG. 1B, which sends operating signals to the motor to control the operation of the motor. For example, the controller 400 may send signals to the motor 220 to: turn the motor on, turn motor the off, and set the operating speed of the motor. Thus, as illustrated in FIG. 1B, the compression system 200 may include a speed sensor 201. The speed sensor 201 may be a motor speed transducer used to determine a rotational speed of the motor 220 and/or a frequency of another reciprocating operation of the compression system 200. For example, a motor speed signal from the motor speed transducer 201 may be provided to the controller 400. The speed sensor or motor speed transducer 201 may, for example, be a Hall effect sensor. The controller 400 may operate the compression system 200 via the motor 220 based on the speed signal and/or any other sensor signal of the oxygen concentrator 100, such as a pressure sensor (e.g., accumulator pressure sensor 107). Thus, as illustrated in FIG. 1B, the controller 400 receives sensor signals, such as a speed signal from the speed sensor 201 and an accumulator pressure signal from the accumulator pressure sensor 107. With such signal(s), the controller 400 may implement one or more control loops (e.g., feedback control) for operation of the compression system 200 based on sensor signals such as accumulator pressure and/or motor speed as described in more detail herein.

The compression system 200 inherently creates substantial heat. Heat is caused by the consumption of power by the motor 220 and the conversion of power into mechanical motion. The compressor 210 generates heat due to the increased resistance to movement of the compressor components by the air being compressed. Heat is also inherently generated due to adiabatic compression of the air by the compressor 210. Thus, the continual pressurization of air produces heat in the enclosure. Additionally, the power supply 180 may produce heat as power is supplied to the compression system 200. Furthermore, users of the oxygen concentrator may operate the device in unconditioned environments (e.g., outdoors) at potentially higher ambient temperatures than indoors, thus the incoming air will already be in a heated state.

Heat produced inside the oxygen concentrator 100 can be problematic. Lithium ion batteries are generally employed as power supplies for oxygen concentrators due to their long life and light weight. Lithium ion battery packs, however, are dangerous at elevated temperatures and safety controls are employed in the oxygen concentrator 100 to shutdown the system if dangerously high power supply temperatures are detected. Additionally, as the internal temperature of the oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator 100. In an implementation, a passive cooling system may be used that takes advantage of the mechanical power produced by the motor 220. Referring to FIGS. 1D and 1E, the compression system 200 includes the motor 220 having an external rotating armature (or external rotatable armature) 230. Specifically, the armature 230 of the motor 220 (e.g. a DC motor) is wrapped around the stationary field that is driving the armature 230. Since the motor 220 is a large contributor of heat to the overall system it is helpful to transfer heat off the motor and sweep it out of the enclosure. With the external high speed rotation, the relative velocity of the major component of the motor 220 and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from the motor 220. The gain in cooling efficiency by mounting the armature 230 externally, allows the elimination of one or more cooling fans, thus reducing the weight and power consumption while maintaining the interior of the oxygen concentrator within the appropriate temperature range. Additionally, the rotation of the externally mounted armature 230 creates movement of air proximate to the motor to create additional cooling.

Moreover, an external rotatable armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to the external rotatable armature 230. In an implementation, the air transfer device 240 is coupled to the external armature 230 such that rotation of the external armature causes the air transfer device to create an air flow that passes over at least a portion of the motor. In an implementation, the air transfer device 240 includes one or more fan blades coupled to the external armature 230. In an implementation, a plurality of fan blades may be arranged in an annular ring such that the air transfer device 240 acts as an impeller that is rotated by movement of the external rotating armature. As depicted in FIGS. 1D and 1E, the air transfer device 240 may be mounted to an outer surface of the external rotatable armature 230, in alignment with the motor 220. The mounting of the air transfer device 240 to the armature 230 allows air flow to be directed toward the main portion of the external rotatable armature 230, providing a cooling effect during use. In an implementation, the air transfer device 240 directs air flow such that a majority of the external rotatable armature 230 is in the air flow path.

Further, referring to FIGS. 1D and 1E, air pressurized by the compressor 210 exits the compressor 210 at the compressor outlet 212. A compressor outlet conduit 250 is coupled to the compressor outlet 212 to transfer the compressed air to the canister system 300. As noted previously, compression of air causes an increase in the temperature of the air. This increase in temperature can be detrimental to the efficiency of the oxygen concentrator. In order to reduce the temperature of the pressurized air, the compressor outlet conduit 250 is placed in the air flow path produced by the air transfer device 240. At least a portion of the compressor outlet conduit 250 may be positioned proximate to the motor 220. Thus, air flow, created by air transfer device 240, may contact both the motor 220 and the compressor outlet conduit 250. In one implementation, a majority of the compressor outlet conduit 250 is positioned proximate to the motor 220. In an implementation, the compressor outlet conduit 250 is coiled around the motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, the compressor outlet conduit 250 can act as a heat exchanger to remove heat that is inherently caused by compression of the air. By removing heat from the compressed air, the number of molecules in a given volume at a given pressure is increased. As a result, the amount of oxygen enriched air that can be generated by each canister during each PSA cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in the canister system 300, the pressure of the exhaust gas being vented from the canisters decreases. The adiabatic decompression of the gas leaving the canister causes the temperature of the exhaust gas to drop as it is vented. In an implementation, the cooled exhaust gas 327 vented from the canister system 300 is directed toward the power supply 180 and toward the compression system 200. In an implementation, a base 315 of canister system 300 receives the exhaust gas 327 from the canisters. The exhaust gas 327 is directed through the base 315 toward the outlet 325 of the base and toward the power supply 180. The exhaust gas 327, as noted, is cooled due to decompression of the gases and therefore passively provide cooling to the power supply 180. When the compression system 200 is operated, the air transfer device 240 will gather the cooled exhaust gas and direct the exhaust gas 327 toward the motor 220 of the compression system 200. The fan 172 may also assist in directing the exhaust gas 327 across the compression system 200 and out of the housing 170. In this manner, additional cooling may be obtained without requiring any further power from the battery.

The oxygen concentrator system 100 may include at least two canisters, each canister including a gas separation adsorbent. The canisters of the oxygen concentrator 100 may be formed from a molded housing. In an implementation, the canister system 300 includes two housing components 310 and 510, as depicted in FIG. 1I. In various implementations, the housing components 310 and 510 of the oxygen concentrator 100 may form a two-part molded plastic frame that defines the two canisters 302 and 304 and the accumulator 106. The housing components 310 and 510 may be formed separately and then coupled together. In some implementations, the housing components 310 and 510 may be injection molded or compression molded. The housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, the housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, the housing components 310 and 510 may be laser or solvent welded together.

As shown, the valve seats 322, 324, 332, and 334 and the conduits 330 and 346 may be integrated into the housing component 310 to reduce the number of sealed connections needed throughout the air flow of the oxygen concentrator 100.

Air pathways/tubing between different sections in the housing components 310 and 510 may take the form of molded conduits. Conduits in the form of molded channels for air pathways may occupy multiple planes in the housing components 310 and 510. For example, the molded air conduits may be formed at different depths and at different positions in the housing components 310 and 510. In some implementations, a majority or substantially all of the conduits may be integrated into the housing components 310 and 510 to reduce potential leak points.

In some implementations, prior to coupling the housing components 310 and 510 together, O-rings may be placed between various points of the housing components 310 and 510 to ensure that the housing components are properly sealed. In some implementations, components may be integrated and/or coupled separately to the housing components 310 and 510. For example, tubing, flow restrictors (e.g., press fit flow restrictors), oxygen sensors, gas separation adsorbents, check valves, plugs, processors, power supplies, etc. may be coupled to the housing components 310 and 510 before and/or after the housing components are coupled together.

In some implementations, apertures 337 leading to the exterior of the housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plugs to seal the passages. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, a spring baffle 139 may be placed into respective canister receiving portions of the housing components 310 and 510 with the spring side of the baffle 139 facing the exit of the canister. The spring baffle 139 may apply force to gas separation adsorbent in the canister while also assisting in preventing gas separation adsorbent from entering the exit apertures. Use of the spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator system 100.

In some implementations, a filter 129 may be placed into respective canister receiving portions of the housing components 310 and 510 facing the inlet of the respective canisters. The filter 129 removes particles from the feed gas stream entering the canisters.

In some implementations, pressurized air from the compression system 200 may enter an air inlet 306. The air inlet 306 is coupled to an inlet conduit 330. Air enters the housing component 310 through the inlet 306, and travels through the inlet conduit 330, and then to the valve seats 322 and 324. FIG. 1J and FIG. 1K depict an end view of the housing component 310. FIG. 1J depicts an end view of the housing component 310 prior to fitting valves to the housing component 310. FIG. 1K depicts an end view of the housing 310 with the valves fitted to the housing component 310. The valve seats 322 and 324 are configured to receive the inlet valves 122 and 124 respectively. The inlet valve 122 is coupled to the canister 302 and the inlet valve 124 is coupled to the canister 304. The housing component 310 also includes the valve seats 332 and 334 configured to receive the outlet valves 132 and 134 respectively. The outlet valve 132 is coupled to the canister 302 and the outlet valve 134 is coupled to the canister 304. The inlet valves 122 and 124 are used to control the passage of air from the conduit 330 to the respective canisters.

In an implementation, pressurized air is sent into one of canisters 302 or 304 while the other canister is being vented. The valve seat 322 includes an opening 323 that passes through the housing component 310 into the canister 302. Similarly, the valve seat 324 includes an opening 375 that passes through the housing component 310 into the canister 304. Air from the inlet conduit 330 passes through the openings 323 or 375 if the respective valves 122 and 124 are open, and enters the respective canisters 302 and 304.

Check valves 142 and 144 (See FIG. 1I) are coupled to the canisters 302 and 304, respectively. The check valves 142 and 144 are one way valves that are passively operated by the pressure differentials that occur as the canisters are pressurized and vented. Oxygen enriched air produced in the canisters 302 and 304 passes from the canisters into the openings 542 and 544 of the housing component 510. A passage (not shown) links the openings 542 and 544 to the conduits 342 and 344, respectively. Oxygen enriched air produced in the canister 302 passes from the canister 302 though the opening 542 and into the conduit 342 when the pressure in the canister 302 is sufficient to open the check valve 142. When the check valve 142 is open, oxygen enriched air flows through the conduit 342 toward the end of the housing component 310. Similarly, oxygen enriched air produced in the canister 304 passes from the canister 304 through the opening 544 and into the conduit 344 when the pressure in the canister 304 is sufficient to open the check valve 144. When the check valve 144 is open, oxygen enriched air flows through the conduit 344 toward the end of the housing component 310.

Oxygen enriched air from either canister 302 or 304 travels through the conduit 342 or 344 and enters the conduit 346 formed in the housing component 310. The conduit 346 includes openings that couple the conduit to the conduit 342, the conduit 344 and the accumulator 106. Thus, oxygen enriched air, produced in the canister 302 or 304, travels to conduit 346 and passes into the accumulator 106.

As illustrated in FIG. 1B, gas pressure within the accumulator 106 may be measured by a sensor, such as with an accumulator pressure sensor 107. (See also FIG. 1F.) Thus, the accumulator pressure sensor 107 generates a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some versions, the pressure sensor 107 may alternatively measure pressure of the gas outside of the accumulator 106, such as in an output path between the accumulator 106 and a valve (e.g., supply valve 160) that controls the release of the oxygen enriched air for delivery to a user in a bolus.

The canister 302 is vented by closing the inlet valve 122 and opening the outlet valve 132. The outlet valve 132 releases the exhaust gas from the canister 302 into the volume defined by the end of the housing component 310. Foam material may cover the end of the housing component 310 to reduce the sound made by release of gases from the canisters. Similarly, the canister 304 is vented by closing the inlet valve 124 and opening outlet the valve 134. The outlet valve 134 releases the exhaust gas from the canister 304 into the volume defined by the end of the housing component 310.

Three conduits are formed in the housing component 510 for use in transferring oxygen enriched air between canisters. As shown in FIG. 1L, the conduit 530 couples the canister 302 to the canister 304. The flow restrictor 151 (not shown) is disposed in the conduit 530, between the canister 302 and the canister 304 to restrict flow of oxygen enriched air during use. The conduit 532 also couples the canister 302 to the canister 304. The conduit 532 is coupled to the valve seat 552 which receives the valve 152, as shown in FIG. 1M. The flow restrictor 153 (not shown) is disposed in the conduit 532, between the canister 302 and the canister 304. The conduit 534 also couples the canister 302 to the canister 304. The conduit 534 is coupled to the valve seat 554 which receives the valve 154, as shown in FIG. 1M. The flow restrictor 155 (not shown) is disposed in the conduit 534, between the canister 302 and the canister 304. The pair of equalization/vent valves 152/154 work with the flow restrictors 153 and 155 to optimize the air flow balance between the two canisters 302 and 304.

Oxygen enriched air in the accumulator 106 passes through the supply valve 160 into the expansion chamber 162 which is formed in the housing component 510. An opening (not shown) in the housing component 510 couples accumulator 106 to the supply valve 160. In an implementation, the expansion chamber 162 may include one or more devices configured to estimate an oxygen concentration of gas passing through the chamber.

An outlet system, coupled to one or more of the canisters, includes one or more conduits for providing oxygen enriched air to a user. In an implementation, oxygen enriched air produced in either of the canisters 302 and 304 is collected in the accumulator 106 through the check valves 142 and 144, respectively, as depicted schematically in FIG. 1B. The oxygen enriched air leaving the canisters 302 and 304 may be collected in the oxygen accumulator 106 prior to being provided to a user. In some implementations, a conduit such as a tube may be coupled to the accumulator 106 to provide the oxygen enriched air to the user. Oxygen enriched air may be provided to the user through an airway delivery device (e.g., a patient interface) that transfers the oxygen enriched air to the user's mouth and/or nose. In an implementation, the airway delivery device may include a tube that directs the oxygen toward a user's nose and/or mouth that may not be directly coupled to the user's nose.

Turning to FIG. 1F, a schematic diagram of an implementation of an outlet system for an oxygen concentrator is shown. A supply valve 160 may be coupled to a conduit to control the release of the oxygen enriched air from the accumulator 106 to the user. In an implementation, supply valve 160 is an electromagnetically actuated plunger valve. The supply valve 160 is actuated by the controller 400 to control the delivery of oxygen enriched air to a user. Actuation of supply valve 160 is not timed or synchronized to the pressure swing adsorption process. Instead, actuation is synchronized to the user's breathing as described below. In some implementations, the supply valve 160 may have continuously-valued actuation to establish a clinically effective amplitude profile for providing oxygen enriched air.

Oxygen enriched air in the accumulator 106 passes through the supply valve 160 into the expansion chamber 162 as depicted in FIG. 1F. In an implementation, the expansion chamber 162 may include one or more devices configured to estimate an oxygen concentration of gas passing through the expansion chamber 162. Oxygen enriched air in the expansion chamber 162 builds briefly, through release of gas from the accumulator 106 by the supply valve 160, and then is bled through a small orifice flow restrictor 175 to a flow rate sensor 185 and then to a particulate filter 187. The flow restrictor 175 may be a 0.025 D flow restrictor. Other flow restrictor types and sizes may be used. In some implementations, the diameter of the air pathway in the housing may be restricted to create restricted gas flow. The flow rate sensor 185 may be any sensor configured to generate a signal representing the rate of gas flowing through the conduit. A particulate filter 187 may be used to filter bacteria, dust, granule particles, etc., prior to delivery of the oxygen enriched air to the user. The oxygen enriched air passes through the filter 187 to the connector 190 which sends the oxygen enriched air to the user via the delivery conduit 192 and to the pressure sensor 194.

The fluid dynamics of the outlet pathway, coupled with the programmed actuations of the supply valve 160, may result in a bolus of oxygen being provided at the correct time and with an amplitude profile that assures rapid delivery into the user's lungs without excessive waste.

The expansion chamber 162 may include one or more oxygen sensors adapted to determine an oxygen concentration of gas passing through the chamber. In an implementation, the oxygen concentration of gas passing through the expansion chamber 162 is estimated using an oxygen sensor 165. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, the oxygen sensor 165 is an ultrasonic oxygen sensor that includes an ultrasonic emitter 166 and an ultrasonic receiver 168. In some implementations, the ultrasonic emitter 166 may include multiple ultrasonic emitters and the ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, an ultrasonic sound wave from emitter 166 may be directed through oxygen enriched air disposed in the chamber 162 to the receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from the emitter 166. This phase shift is due to the relatively slow velocity of sound through a gas medium as compared with the relatively fast speed of the electronic pulse through wire. The phase shift, then, is proportional to the distance between the emitter 166 and the receiver 168 and inversely proportional to the speed of sound through the expansion chamber 162. The density of the gas in the chamber 162 affects the speed of sound through the expansion chamber 162 and the density is proportional to the ratio of oxygen to nitrogen in the expansion chamber 162. Therefore, the phase shift can be used to measure the concentration of oxygen in the expansion chamber 162. In this manner the relative concentration of oxygen in the accumulator 106 may be estimated as a function of one or more properties of a detected sound wave traveling through the accumulator 106.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic oxygen sensor system may be increased by increasing the distance between the emitter 166 and the receiver 168, for example to allow several sound wave cycles to occur between the emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of the expansion chamber 162 may be reduced or cancelled. The shift caused by a change of the distance between the emitter 166 and the receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. patent application Ser. No. 12/163,549, entitled “Oxygen Concentrator Apparatus and Method”, which published as U.S. Publication No. 2009-0065007 on Mar. 12, 2009 and is incorporated herein by reference.

The flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. The flow rate sensor 185 may be coupled to the controller 400. The rate of gas flowing through the outlet system may be an indication of the breathing volume of the user. Changes in the flow rate of gas flowing through the outlet system may also be used to determine a breathing rate of the user. The controller 400 may generate a control signal or trigger signal to control actuation of the supply valve 160. Such control of actuation of the supply valve may be based on the breathing rate and/or breathing volume of the user, as estimated by the flow rate sensor 185.

In some implementations, the ultrasonic oxygen sensor 165 and, for example, the flow rate sensor 185 may provide a measurement of an actual amount of oxygen being provided. For example, the flow rate sensor 185 may measure a volume of gas (based on flow rate) provided and the ultrasonic oxygen sensor 165 may provide the concentration of oxygen of the gas provided. These two measurements together may be used by the controller 400 to determine an approximation of the actual amount of oxygen provided to the user.

Oxygen enriched air passes through the flow rate sensor 185 to the filter 187. The filter 187 removes bacteria, dust, granule particles, etc. prior to providing the oxygen enriched air to the user. The filtered oxygen enriched air passes to the connector 190. The connector 190 may be a “Y” connector coupling the outlet of the filter 187 to the pressure sensor 194 and the delivery conduit 192. The pressure sensor 194 may be used to monitor the pressure of the gas passing through the delivery conduit 192 to the user. In some implementations, the pressure sensor 194 is configured to generate a signal that is proportional to the amount of positive or negative pressure applied to a sensing surface. Changes in pressure, sensed by the pressure sensor 194, may be used to determine a breathing rate of a user, as well as the onset of inhalation (also referred to as the trigger instant) as described below. The controller 400 may control actuation of the supply valve 160 based on the breathing rate and/or onset of inhalation of the user. In an implementation, the controller 400 may control actuation of the supply valve 160 based on information provided by either or both of the flow rate sensor 185 and the pressure sensor 194.

Oxygen enriched air may be provided to a user through the delivery conduit 192. In an implementation, the conduit 192 may be a silicone tube. The conduit 192 may be coupled to a user using an airway delivery device 196, as depicted in FIGS. 1G and 1H. The airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. The nasal cannula airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to the oxygen concentrator 100. The mouthpiece 198 may be the only device used to provide oxygen enriched air to the user, or a mouthpiece may be used in combination with a nasal delivery device 196 (e.g., a nasal cannula). As depicted in FIG. 1H, oxygen enriched air may be provided to a user through both the nasal cannula airway delivery device 196 and the mouthpiece 198.

The mouthpiece 198 is removably positionable in a user's mouth. In one implementation, the mouthpiece 198 is removably couplable to one or more teeth in a user's mouth. During use, oxygen enriched air is directed into the user's mouth via the mouthpiece. The mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to the mouthpiece 198 when a change in pressure is detected proximate to the mouthpiece. In one implementation, the mouthpiece 198 may be coupled to a pressure sensor 194. When a user inhales air through the user's mouth, the pressure sensor 194 may detect a drop in pressure proximate to the mouthpiece. The controller 400 of the oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, the oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, the mouthpiece 198 is used in combination with the nasal cannula airway delivery device 196 to provide oxygen enriched air to a user, as depicted in FIG. 1H. Both the mouthpiece 198 and the nasal airway delivery device 196 are coupled to an inhalation sensor. In one implementation, the mouthpiece 198 and the nasal cannula airway delivery device 196 are coupled to the same inhalation sensor. In an alternate implementation, the mouthpiece 198 and the nasal cannula airway delivery device 196 are coupled to different inhalation sensors. In either implementation, the inhalation sensor(s) may detect the onset of inhalation from either the mouth or the nose. The oxygen concentrator 100 may be configured to provide oxygen enriched air to the delivery device (i.e. mouthpiece 198 or nasal cannula airway delivery device 196) proximate to which the onset of inhalation was detected. Alternatively, oxygen enriched air may be provided to both the mouthpiece 198 and the nasal cannula airway delivery device 196 if onset of inhalation is detected proximate either delivery device. The use of a dual delivery system, such as depicted in FIG. 1H may be particularly useful for users when they are sleeping and may switch between nose breathing and mouth breathing without conscious effort.

Operation of oxygen concentrator 100 may be performed automatically using the internal controller 400 coupled to various components of the oxygen concentrator 100, as described herein. The controller 400 includes one or more processors 410 and internal memory 420, as depicted in FIG. 1B. Methods used to operate and monitor oxygen concentrator 100 may be implemented by program instructions stored in the internal memory 420 or an external memory medium coupled to the controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs are executed, or may be located in an external computing device that connects to the controller 400 over a network, as detailed below. In the latter instance, the external computing device may provide program instructions to the controller 400 for execution. The term “memory medium” may include two or more memory media that may reside in different locations, e.g., in different computing devices that are connected over a network.

In some implementations, the controller 400 includes the processor 410 that includes, for example, one or more field programmable gate arrays (FPGAs), microcontrollers, etc. included on a circuit board disposed in the oxygen concentrator 100. The processor 410 is configured to execute programming instructions stored in the memory 420. In some implementations, programming instructions may be built into the processor 410 such that a memory external to the processor 410 may not be separately accessed (i.e., the memory 420 may be internal to the processor 410).

The processor 410 may be coupled to various components of oxygen concentrator 100, including, but not limited to the compression system 200, one or more of the valves used to control fluid flow through the system (e.g., valves 122, 124, 132, 134, 152, 154, 160), the oxygen sensor 165, the pressure sensor 194, the flow rate sensor 185, temperature sensors (not shown), the fan 172, and any other component that may be electrically controlled. In some implementations, a separate processor (and/or memory) may be coupled to one or more of the components.

The controller 400 is configured (e.g. programmed by program instructions) to operate oxygen concentrator 100 and is further configured to monitor the oxygen concentrator 100 such as for malfunction states or other process information. For example, in one implementation, the controller 400 is programmed to trigger an alarm if the system is operating and no breathing is detected by the user for a predetermined amount of time. For example, if the controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for the controller 400 to reset this alarm function. Alternatively, if the system is accidentally left on when the delivery conduit 192 is removed from the user, the alarm may serve as a reminder for the user to turn the oxygen concentrator 100 off.

The controller 400 is further coupled to the oxygen sensor 165, and may be programmed for continuous or periodic monitoring of the oxygen concentration of the oxygen enriched air passing through the expansion chamber 162. A minimum oxygen concentration threshold may be programmed into the controller 400, such that the controller 400 lights an LED visual alarm and/or an audible alarm to warn the user of the low concentration of oxygen.

The controller 400 is also coupled to the internal power supply 180 and may be configured to monitor the level of charge of the internal power supply. A minimum voltage and/or current threshold may be programmed into the controller 400, such that the controller 400 lights an LED visual alarm and/or an audible alarm to warn the user of low power condition. The alarms may be activated intermittently and at an increasing frequency as the battery approaches zero usable charge. Further functions that may be implemented with or by the controller 400 are described in detail in other sections of this disclosure. The controller 400 may be communicatively coupled to one or more external devices to make up a connected oxygen therapy system. The one or more external devices may be a remote external device. The one or more external devices may be an external computing device to make up a connected oxygen therapy system.

FIG. 1N illustrates one implementation of a connected oxygen therapy system, in which the controller 400 may include a Cellular Wireless Module such as the transceiver 430, or other wireless communications module, configured to allow the controller 400 to communicate, using a wireless communication protocol such as the Global System for Mobile Telephony (GSM) or other protocol (e.g., WIFI), with a remote computing device 460 such as over a network. The computing device (or a remote external device 460) such as a cloud-based server 460 (or server 460) may exchange data with the controller 400. The remote external device may be a remote computing device, such as a portable computing device. For instance, the controller 400 may also include a short range wireless module 440 (SRWM 440) configured to enable the controller 400 to communicate, using a short range wireless communication protocol such as Bluetooth, with a portable computing device 480 such as a smartphone 480. The smartphone 480 may be associated with a user of the POC 100′ in FIG. 4 or the POC 100 in FIG. 1. When there are two or more external devices, each external device may be the same or different. For example, when there are two external devices and each external device is the same, there may be two servers 460. Alternatively, when there are two external devices and each external device is different, there may be a server 460 and a portable computing device 480.

The server 460 may also be in wireless communication with the portable computing device 480 using a wireless communication protocol such as GSM. A processor of the smartphone 480 may execute a program known as an “app” to control the interaction of the smartphone 480 with the POC 100′ and/or the server 460.

The server 460 may also be in communication with a personal computing device 464 via a wired or wireless connection to a wide-area network such as the Internet, or a local-area network such as an Ethernet. A processor of the personal computing device 464 may execute a “client” program to control the interaction of the personal computing device 464 with the server 460. One example of a client program is a browser.

Further functions that may be implemented with or by the controller 400 are described in detail in other sections of this disclosure.

The control panel 600 serves as an interface between a user and the controller 400 to allow the user to initiate predetermined operation modes of the oxygen concentrator 100 (or oxygen concentrator 100′) and to monitor the status of the system. FIG. 1O depicts an implementation of the control panel 600. Charging input port 605, for charging the internal power supply 180, may be disposed in control panel 600.

In some implementations, the control panel 600 may include buttons to activate various operation modes for the oxygen concentrator 100. For example, the control panel 600 may include a power button 610, flow rate setting buttons 620 to 626, an active mode button 630, a sleep mode button 635, an altitude button 640, and a battery check button 650. In some implementations, one or more of the buttons may have a respective LED that may illuminate when the respective button is pressed, and may power off when the respective button is pressed again. The power button 610 may power the system on or off. If the power button 610 is activated to turn the system off, the controller 400 may initiate a shutdown sequence to place the system in a shutdown state (e.g., a state in which both canisters are pressurized). The flow rate setting buttons 620, 622, 624, and 626 allow the prescribed continuous flow rate of oxygen enriched air to be selected (e.g., 0.2 LPM by the button 620, 0.4 LPM by the button 622, 0.6 LPM by the button 624, and 0.8 LPM by the button 626 In other implementations, the number of flow rate settings may be increased or decreased. After a flow rate setting is selected, oxygen concentrator 100 will then control operations to achieve production of the oxygen enriched air according to the selected flow rate setting. The altitude button 640 may be activated when a user is going to be in a location at a higher elevation than the oxygen concentrator 100 is regularly used by the user.

The battery check button 650 initiates a battery check routine in the oxygen concentrator 100 which results in a relative battery power remaining LED 655 being illuminated on the control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically from the detected breathing rate or depth, and/or the user may manually indicate an active mode or a sleep mode by pressing the button 630 for active mode or the button 635 for sleep mode respectively. In some implementations, the POC 100 defaults to active mode.

The main use of an oxygen concentrator 100 is to provide supplemental oxygen to a user. One or more flow rate settings may be selected on a control panel 600 of the oxygen concentrator 100, which then will control operations to achieve production of the oxygen enriched air according to the selected flow rate setting. In some versions, a plurality of flow rate settings may be implemented (e.g., five flow rate settings). As described in more detail herein, the controller 400 may implement a POD (pulsed oxygen delivery) or demand mode of operation. Controller 400 may regulate the size of the one or more released pulses or boluses to achieve delivery of the oxygen enriched air according to the selected flow rate setting.

In order to maximize the effect of the delivered oxygen enriched air, the controller 400 may be programmed to synchronize the release of each bolus of the oxygen enriched air with the user's inhalations. Releasing a bolus of oxygen enriched air to the user as the user inhales may reduce wastage of oxygen by not releasing oxygen, for example, when the user is exhaling. The flow rate settings on the control panel 600 may correspond to minute volumes (bolus volume multiplied by breathing rate per minute) of delivered oxygen, e.g. 0.2 LPM, 0.4 LPM, 0.6 LPM, 0.8 LPM, 1 LPM, 1.1 LPM.

Oxygen enriched air produced by oxygen concentrator 100 may be stored in the oxygen accumulator 106 and, in a POD mode of operation, released to the user as the user inhales. The amount of oxygen enriched air provided by the oxygen concentrator 100 is controlled, in part, by the supply valve 160. In an implementation, the supply valve 160 is opened for a sufficient amount of time to provide the appropriate amount of oxygen enriched air, as estimated by controller 400, to the user. In order to minimize the wastage of oxygen, the oxygen enriched air may be released as a bolus soon after the onset of a user's inhalation is detected. For example, the bolus of oxygen enriched air may be released in the first few milliseconds of a user's inhalation.

In an implementation, an inhalation sensor such as a pressure sensor 194 may be used to detect the onset of inhalation by the user (a process referred to as “triggering”). For example, the onset of the user's inhalation may be detected by using the pressure sensor 194. In use, the delivery conduit 192 for providing oxygen enriched air is coupled to a user's nose and/or mouth through the nasal airway delivery device 196 and/or the mouthpiece 198. The pressure in the delivery conduit 192 is therefore representative of the user's airway pressure and hence indicative of user respiration. At the onset of inhalation, the user begins to draw air into their body through the nose and/or mouth. As the air is drawn in, a negative pressure is generated at the end of the delivery conduit 192, due, in part, to the venturi action of the air being drawn across the end of the delivery conduit 192. The controller 400 analyzes the pressure signal from the pressure sensor 194 to detect a drop in pressure indicating the onset of inhalation. Upon detection of the onset of inhalation, the supply valve 160 is opened to release a bolus of oxygen enriched air from the accumulator 106.

A positive change or rise in the pressure in the delivery conduit 192 indicates an exhalation by the user. The controller 400 may analyze the pressure signal from pressure sensor 194 to detect a rise in pressure indicating the onset of exhalation. In one implementation, when a positive pressure change is sensed, the supply valve 160 is closed until the next onset of inhalation is detected. Alternatively, the supply valve 160 may be closed after a predetermined interval known as the bolus duration. By measuring the intervals between adjacent onsets of inhalation, the user's breathing rate may be estimated. By measuring the intervals between onsets of inhalation and the subsequent onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through the delivery conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to controller 400 in the oxygen concentrator 100 electronically via a wire or through telemetry such as through Bluetooth™ or other wireless technology.

The sensitivity of the triggering process is governed by a trigger threshold. The signal from the pressure sensor 194 is compared with the trigger threshold to determine whether a significant drop in pressure has taken place, thereby indicating onset of inhalation. Adjusting the trigger threshold alters the sensitivity of the triggering process. In some implementations, the trigger threshold is set to give the triggering process a higher sensitivity when the POC 100 is in sleep mode (e.g. as estimated automatically or as requested by the user via the sleep mode button 635) compared to when the POC 100 is in active mode (e.g. as estimated automatically or as requested by the user via the active mode button 630).

The POC 100 is in active mode and an onset of inhalation has not been detected for a predetermined interval, e.g. 8 seconds, the POC 100 changes to sleep mode, which increases the trigger sensitivity as described above. If onset of inhalation is not detected for a further predetermined interval (e.g. 8 seconds), the POC 100 enters “auto-pulse” mode. In auto-pulse mode, the controller 400 controls actuation of the supply valve 160 so as to deliver boluses at regular, predetermined auto-pulse intervals, e.g. 4 seconds. The POC 100 exits auto-pulse mode once onset of inhalation is detected by the triggering process or the POC 100 is powered off.

In some implementations, if the user's current activity level, such as that estimated using the detected user's breathing rate, exceeds a predetermined threshold, controller 400 may implement an alarm (e.g., visual and/or audio) to warn the user that the current breathing rate is exceeding the delivery capacity of the oxygen concentrator 100. For example, the threshold may be set at 40 breaths per minute (BPM).

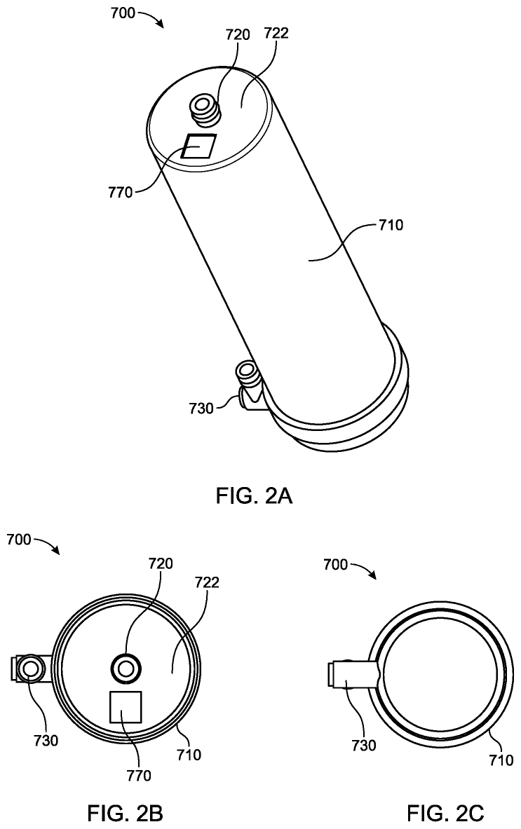

FIG. 2A is a perspective view of the components of an example of a modular sieve bed assembly 700 that may be used for adsorption in an oxygen concentrator, such as the portable oxygen concentrator 100′ (or POC 100′) in FIG. 3. The modular sieve bed assembly 700 is a component that may be replaced in the POC 100′ in FIG. 3. The modular sieve bed assembly 700 serves the same adsorption functions as the sieve bed assembly 300 in the POC 100 explained above. FIG. 2B is a top view of the sieve bed assembly 700 and FIG. 2C is a bottom view of the sieve bed assembly 700. The sieve bed assembly 700 is modular and removable. Advantageously, the sieve bed assembly 700 is removable by a user. As such, the adsorbent material may be replaced and the sieve bed assembly 700 may be repeatedly used. The sieve bed assembly 700 is built as an independent unit and includes a canister 710 (or sieve bed 710) having an intake/inlet port 720 and an outlet port 730 on an opposite end, wherein the sieve bed assembly 700 is pneumatically interfaced to the POC 100′. The sieve bed assembly 700 of the POC 100′ may have more than one canister 710. For instance, the sieve bed assembly 700 of the POC 100′ may comprise two canisters 710 similar to the sieve bed assembly 300 of the POC 100.

In this example, an identification device 770 is attached to the canister 710 for purposes of identifying the sieve bed assembly 700 for tracking purposes. In this example, the identification device 770 is attached on the panel 722 of the canister 710. However, it is to be understood that the identification device 770 may be attached to any exterior surface at any location that allows the identification device 770 to be read. Alternatively, the identification device 770 may be embedded in one of the components of the sieve bed assembly 700. The identification device 770 may also be adhered to any exterior or interior surface of the sieve bed assembly 700. A similar identification device may also be attached to the sieve bed assembly 300 of the POC 100 described above for similar functions as described herein.

In this example, the identification device 770 is a RFID tag that includes identification data, such as a unique serial number, associated with the sieve bed assembly 700 and no other sieve bed assembly. Alternatively, the identification device 770 may be operable to communicate the identification data using another communication protocol such as NFC. Other information may also be stored in the identification device 770 such as manufacturing information. The manufacturing information includes (but is not limited to) manufacture date, expiration date, component part number, component revision number, manufacture location and refill count. As used herein, the term “component part number” refers to an identifier of a particular component in the POC 100′. The identifier may comprise at least one number, at least one alphabetic character or a combination thereof. As used herein, the term “component revision number” refers to an identifier that reflects the current revision of the particular component. The identifier may comprise at least one number, at least one alphabetic character or a combination thereof. As used herein, the term “refill count” refers to the number of times that the sieve bed assembly 700 has been refilled, whereby the sieve bed assembly 700 may be refilled by removing the adsorbent material within the canister 710 and replacing with fresh adsorbent material. The fresh adsorbent material may be new adsorbent material or regenerated adsorbent material. Advantageously, the canister 710 may be refilled multiple times, thereby leading to a savings in costs and making the sieve bed assembly 700 more environmentally friendly.

The identification device 770 may optionally comprise an authentication code (or an authentication identifier), thereby preventing or reducing counterfeiting. The authentication code of the identification device 770 may be invisible to human eyes in the form of a signal or may comprise visible yet unreadable to humans, indicia, symbols, or code. Alternatively, certain graphics may be readable by humans as well as scanned. When in use, the identification device 770 may be scanned/read using the reader. Once the indicia, symbols, or code is detected, it is communicated to the remote external device, such as the server 460. The server 460 may include a database, which is searched for a match for the detected identification information from the signal, indicia, symbols or code. If there is a match with the identification information from the signal, indicia, symbols or code in the database maintained by, for instance, the manufacturer or administrator, the sieve bed assembly 700 is deemed as a genuine product. If there is no match, then the sieve bed assembly 700 is deemed to be an imitation or a knockoff. The reader and tag can be configured to make use of additional security measures such as a checksum, thereby preventing or reducing counterfeiting of the identification information. As used herein, the term “checksum” refers to any checksum, cyclic redundancy code, digest, data structure or other compressed representation of data. For instance, a reader may be configured to compute a checksum or other representation of an identifier received and store the representation in a tag associated with a particular sieve bed assembly. To detect whether a replacement sieve bed assembly 700 is a genuine product, the reader may read the identifier of a replacement sieve bed assembly 700, compute a second representation, and then compare the more recently computed representation to the representation of the previous sieve bed assembly 700. If the two representations match, the reader can be relatively confident that the replacement sieve bed assembly 700 is genuine.

In this example, the controller 400 may activate a reader to read the identification device 770 when the sieve bed assembly 700 is installed in the oxygen concentrator 100. The reader may be mounted on a printed circuit board assembly (PCBA) for the oxygen concentrator 100 or may be a stand-alone reader. The reader is configured such that it can read the identification device 770, thereby obtaining the unique serial number associated with the sieve bed assembly 710 and any other information. Consequently, when the reader is mounted on the PCBA, the PCBA comprises the reader, the printed circuit board and the controller 400, wherein the reader is operable to read the identification information (or identification data) from the identification device 770 and wherein the controller is operable to read the identification information (or identification data) and transmit the read identification information (or identification data) via a transceiver.

FIG. 3 is a block diagram of a reader system 350 of an oxygen concentrator such as the oxygen concentrator 100′ that obtains the identification data from sieve bed assemblies such as the sieve bed assembly 700. This process may be performed automatically under the command of a controller similar to the controller 400 described above. The reader system 350 includes the identification device 770 on the sieve bed assembly 700 and a printed circuit board 360 of the oxygen concentrator 100′ that includes a remote RFID reader 362 and a controller 364. Similar to the POC 100 and controller 400 explained above, the operation of the oxygen concentrator 100′ is controlled by the controller 364 that is also mounted on the printed circuit board 360. Consequently, the printed circuit board assembly (PCBA) comprises the remote RFID reader 362, the printed circuit board 360 and the controller 364. In this example the PCBA also includes a memory 368 with similar functions to the memory 420 in FIG. 1A and a transceiver 366 with similar functions to the transceiver module 430 in FIG. 1A. The RFID reader 362 is coupled to an antenna 370.

The controller 364 in this example may power the RFID reader 362 to send an activation signal via the antenna 370. The activation signal is received by the identification device 770 (such as RFID device 770) and triggers an internal transmitter to send the identification data to the antenna 370. The reader 362 receives the identification data from the antenna 370. The controller 364 can read the identification data and store the data in the memory 368 and/or cause the identification data to be transmitted externally through the transceiver module 366 and/or provide processing of the identification data. The reader 362 may provide initial processing of the identification data. When the reader 362 provides initial processing of the identification data, the controller 364 may provide final processing of the identification data.

Alternatively, a readable/writable storage device such as an EEPROM 780 may be installed on the sieve bed 700 in combination with the RFID tag 700 or in place of the RFID tag 700 to store additional data relating to the sieve bed 700 and its operational history. The EEPROM 780 may also be integrated into integrated electronics of the RFID tag 700. The EEPROM 780 may be programmed with run time, effective capacity, remaining life, imbalance parameters, and other information relating to the sieve bed described above. Any POC may access the data on the EEPROM 780 through a physical connector or a wireless transceiver. Thus, if the sieve bed 700 is removed from an initial POC and moved to another POC, the second POC will read the data on the EEPROM 780 and with the sieve bed will be able to use the sieve bed instantly with the right configuration. During the manufacturing process, other information may be written into the storage device 780.

The EEPROM 780 may store the identification information described above. However, more data may be stored to provide a more detailed, and therefore more difficult to copy identification information. For example, an encryption key that authenticates the sieve bed to prevent fraudulent usage of “self-filled” zeolite replacement may also be stored in the EEPROM 780. Increased security may be provided by the relatively larger memory capacity of the EEPROM 780 allowing for the storage of a key having more bits. When retrieving a malfunctioning or damaged sieve bed, the EEPROM 780 may store relevant information of issues relating to the sieve bed 700 that the controller of the POC can detect. Data on the performance of the sieve bed 700 may be recorded on the EEPROM 780 to assist in the diagnosis of the malfunction or damage. Additional identification information may be stored to designate the start of events such as a warranty period.

The reader system 350 can optionally detect whether the sieve bed assembly 700 is properly installed in the retention mechanism of the oxygen concentrator 100′. In this example, the retention mechanism is a spring loaded plunger and a corresponding mating retention mechanism is a slot in the sieve bed assembly 700, wherein the slot is adapted to mate with the spring loaded plunger. The location of the identification tag device 770 may be selected in relation to the printed circuit board 360 and the distance may be correlated to the strength of the signal received by the tag device 770. A strong signal may indicate that the sieve bed assembly 700 is properly installed in the retention mechanism of the oxygen concentrator 100′. Alternatively, the sieve bed assembly 700 and location of the tag device 770 may be selected so that the tag device 770 can only be activated by the reader 362 when the sieve bed assembly 700 is properly installed in the retention mechanism of the oxygen concentrator 100′. Other retention mechanisms may include an assembly where either the sieve bed assembly 700 or oxygen concentrator 100′ includes a screw operated tab and the corresponding mating retention mechanism includes a slot which mates with the tab. The tab is rotated into the slot when the sieve bed assembly 700 is mated to a platform of the oxygen concentrator 100′, thereby retaining the sieve bed assembly 700. Conversely, reverse rotation of the tab releases the sieve bed assembly 700. When the tag device 770 is an NFC tag and the reader 362 is an NFC reader, the quality of the NFC signal from the NFC reader can be correlated to the strength of the NFC signal received by the NFC reader 362. As such, the strength of the NFC signal received by the NFC reader 362 may allow the system to detect whether the sieve bed assembly 700 is properly installed.

In this example, the identification data is obtained from the tag device 770 by the controller 364 through the internally installed reader 362 on the oxygen concentrator 100′. Alternatively, the tag device 770 may also be read at the factory when the sieve bed assembly 700 is manufactured. In this example, a dedicated reader device on the assembly line or operated by a user may be employed to read the identification data. Alternatively, an external reader may be used to read the identification data when the sieve bed assembly 700 is mated with the oxygen concentrator 100′. Thus, a label may be used for the tag device 770 that includes a visibly readable code, symbol or indicia, such as a bar code or a QR code that includes the identification data of the sieve bed assembly 700. The use of such a code may thus use a portable computing device such as a smart phone with a camera to read the label.

Once the identification data is obtained from the sieve bed assembly 700 and recorded, the age of the sieve bed assembly 700 may be tracked. Thus, adsorbent materials such as the zeolite in the sieve bed assembly 700 may be monitored in relation to operational data collected by the controller 400 of the POC 100′. For example, O₂ purity data collected by the controller 400 may be correlated with the identified sieve bed assembly 700 and its manufacturing information. Thus, analysis may be performed on why sieve bed assemblies for a certain batch are degrading faster than others. Alternatively, defects may be determined and correlated to a certain batch. Further, such operational data may be gathered and correlated to an individual sieve bed assembly 700. Such data may be useful for analysis of manufacturing processes and servicing processes. Results of such analysis may be used to make predictive maintenance more accurate, and warn a patient that a sieve bed assembly 700 of their POC is nearing the end of life. Furthermore, the operational data (such as O₂ purity data) may help determine the effectiveness of a design change, such as to the sieve bed assembly 700, the POC 100′ or the software described herein, wherein the purpose of the design change is to improve the sieve bed assembly 700. For instance, the improvements to the sieve bed assembly 700 may be related to increasing the shelf life (or longevity) of the sieve bed assembly 700 and/or counterfeit detection/preventing. Advantageously and as described above, the tag device 770 may be a means of preventing or reducing counterfeiting.

The use of the identification device for tracking of the use of the sieve bed assembly 700 in POCs such as the POC 100′ or POC 100 allows the identification data to be obtained in a fast manner. Furthermore, use of a reader makes the method of obtaining the identification data more accurate compared to manual input of the identification data. Beside these advantages of speed and accuracy, there are other advantages in terms of cost, scalability, ease of use and convenience.

Information that the sieve bed assembly 700 has been actually installed allows the identification information to be linked to a particular POC and patient. This assists in confirming that a patient is using the POC properly and allows manufacturers to determine the accuracy of warranty claims.

FIG. 4 illustrates one implementation of a POC component monitoring system 450, in which the controller 364 of the POC 100′ includes the transceiver module 366 configured to allow the controller 364 to communicate, using a wireless communication protocol such as the Global System for Mobile Telephony (GSM) or other protocol (e.g., WIFI), with a remote external device (or remote computing device) such as the cloud-based server 460 over a network 470. The network 470 may be a wide-area network such as the Internet, or a local-area network such as an Ethernet. Alternatively, or in addition, the remote external device may be a remote computing device, such as a portable computing device. For instance, the controller 364 may also include a short range wireless module in the transceiver module 366 configured to enable the controller 364 to communicate, using a short range wireless communication protocol such as Bluetooth™, with the portable computing device 480 such as a smartphone. The smartphone 480 may be associated with a user 1000 of the POC 100′.

The server 460 may also be in wireless communication with the portable computing device 480 using a wireless communication protocol such as GSM. A processor of the smartphone 480 may execute a program 482 known as an “app” to control the interaction of the smartphone with the POC 100′ and/or the server 460. In this example, when the POC 100′ first is activated, the controller 364 sends the identification data read by the reader 362 in FIG. 3 relating to the sieve bed assembly 700 in the POC 100′. The identification data will allow other data relating to the sieve bed assembly 700 and the operation of the POC 100′ to be correlated for different operations.

The server 460 includes an analysis engine 462 that may execute operations such as a component service date prediction algorithm and a servicing routine. The server 460 may also be in communication with other devices such as a personal computing device workstation 464 via a wired or wireless connection via the network 470. A processor of the personal computing device 464 may execute a “client” program to control the interaction of the personal computing device 464 with the server 460. One example of a client program is a browser. The server 460 has access to a database 466 that stores operational data about the POCs and users managed by the system 450. The database 466 may be segmented into individual databases such as a user database having information about users of the POCs and operational data associated with the POC use and corresponding sieve bed assembly use by the respective users, a manufacturer database including manufacturing information of the sieve bed assemblies, transportation and storage information, and a reference database including deterioration curves, common profiles, and default servicing times. The deterioration curves could include, but are not limited to, time profiles of: oxygen purity output from the sieve bed assemblies, remaining capacity of the sieve bed assemblies, characteristic pressure delivered by the compressor, flow rate output of the POC, internal humidity of the POC, and so on. Servicing times may be categorized by the expected overall lifetime of the sieve bed assembly with additional information in relation to the amount of use of the components in the POC. The server 460 may also be in communication via the network 470 with servers operated by other enterprises such as a supplier server 468 (or supply entity server 468) that coordinates the ordering and supply of replacement sieve bed assemblies for POCs.

The user 1000 of the POC 100′ and portable computing device 480 may be organized as a POC user system 490. The connected oxygen concentrator component monitoring system 450 may comprise a plurality or “fleet” of POC user systems 490, 492, 494 and 496 that each include a POC user, POCs such as the POC 100′ or the POC 100, and portable computing devices such as the portable computing device 480. Each of the other POC user systems 492, 494 and 496 are in communication with the server 460, either directly or via respective portable computing devices associated with respective users of the POCs. Corresponding controllers such as the controller 400 and transceivers such as the transceiver 430 in each of the POCs of the systems 492, 494 and 496 read the identification data from sieve bed assemblies and send such data to the server 460. The workstation such as the personal computing device 464 may be associated with a component supplier and or a health management entity (HME) that is responsible for the therapy of a population of users of the fleet of POCs.

Predictions of remaining usage times of sieve bed assemblies may be utilized by the various entities in the component monitoring system 450. In one implementation, the application 482 running on the portable computing device 480 could cause predicted values of remaining usage times of various POC components to be displayed on a display of the portable computing device 480. This could occur on the instruction of the server 460 via a “push notification” to the app, or on the initiative of the app itself.

In a further implementation, the server 460 may be configured to host a portal system. The portal system may receive, from the personal computing device 464 or directly from the POC 100′, data relating to the operation of the POC 100′. For example, such operational data may include estimates of remaining capacity of sieve bed assemblies in a POC 100′. As described above, the personal computing device 464 may execute a client application such as a browser to allow a user of the personal computing device 464 (such as a representative of an HME) to access the operational data of the POC 100′, and other POCs in a connected oxygen therapy system 450, via the portal system hosted by the server 460. In this fashion, such a portal system may be utilized by an HME to manage a population of users of POC devices, e.g. the POC device 100′, or POC user systems 492, 494, and 496 in the connected oxygen therapy system 450. The HME may allow the data server 460 (or server 460) to provide supply information, such as the type of component, address of the user, convenient time of service, the ability or willingness of the user to do the service themselves, etc., on the fleet of POC devices to service entities by communicating component supply data to the supply entity server 468.

The portal system may provide actionable insights into user or device condition for the population of POC devices and their users based on the operational data received by the portal system. Such insights may be based on rules that are applied to the operational data. In one implementation, the predicted remaining usage times of a fleet of POCs may be displayed to a representative of an HME on a display of a personal computing device 464 in a “window” of a client program interacting with the portal system. Further, a rule may be applied to each remaining usage time prediction based on the status of different components. One example of such a rule is “If the remaining usage time for a POC is less than three weeks, highlight the POC in the display of remaining usage times”. Application of such a rule to the remaining usage times results in the highlighting on the display of POCs with sieve bed assemblies approaching exhaustion or compressors near wearing out. The highlighted POCs may then be noted by the HME for imminent servicing. This is one example of the kind of rule-based fleet management made possible by a routine of predicting remaining component usage times operating within the connected oxygen therapy system 450.

Based on the identification data from the sieve bed assemblies and collected operational data, predictions may be made by a routine executed by the server 460 predicting the lifetime of the sieve bed assemblies. Optionally, such as in case where the POC 100′ determines an estimate of the remaining capacity of a sieve bed assembly, the POC 100′ may communicate a message, which may be based on the estimate, such as by a comparison with a threshold (e.g., if the estimate is at or below a threshold), to an external computing device of the system 450 such as to provide a notification message of a need for a replacement sieve bed assembly for the POC 100′. Such a message may comprise a request for a new sieve bed assembly such as for arranging a purchase or replacement order for a new sieve bed assembly via an ordering or fulfillment system implemented with any of the devices of FIG. 4 such as the supply entity server 468. Such a message may also be generated by any of the devices of the system 450 that receives either the remaining capacity estimate or the measurements and parameters necessary for determining the estimate. In such a case, the message may be further transmitted to other systems, such as a purchasing, ordering or fulfillment system or server(s) that may be configured to communicate with a device of the system 450 for arranging and/or completing such orders. Still further, in some versions, the POC may make a change in a control parameter of the POC based on the estimate or a comparison of the estimate and one or more thresholds. For example, one or more parameters for control of the PSA cycle of the POC may be adjusted based on the comparison. Such adjustments may include, for example, to parameters for the various valve timings of the valves that control flow through the canisters for feed and purge cycles and/or compressor speed, etc. Such adjustments may be implemented for increasing remaining sieve bed assembly usage time if a partially exhausted sieve bed assembly is detected (e.g., less than 70%, 50% etc.) or resuming normal operating parameters for a detection of a replaced sieve bed assembly (e.g., greater than 50% or at or near 100%).

The system 450 also allows predictions of service dates for servicing sieve bed assemblies of entire groups of POCs of the fleet of POCs monitored by the system 450 based the correlation of identification data of the sieve bed assemblies and the operational data. Such economies of scale provide better servicing for the POC fleet managed by the system 450 in relation to sieve bed assembly replacement. Many HMEs or service businesses manage fleets of POCs in geographically disparate locations. This could be POC users spread across a state or nationally, or users in isolated areas that are expensive to access. By anticipating when individual POCs within are fleet are going to need to be serviced, it is possible to ‘cluster’ servicing to minimize staff and/or transportation costs. For example, POC A's sieve bed assembly may be going to fail in 5 days, POC B's in 4 weeks and POC C's sieve bed assembly in 7 weeks. Rather than servicing each POC individually in the days before failure (and making three trips), a business owner may choose to service all three at the same time because they are geographically distant but clustered near each other, and the salaried costs of the technician outweigh the costs of the replacement sieve bed assemblies. When this logic is applied to fleets of tens of thousands of POCs the efficiency gains are significant.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A method of tracing a sieve bed assembly for installation in an oxygen concentrator, the method comprising:
attaching an identification device including identification data relating to a sieve bed assembly to the sieve bed assembly, wherein the identification data is capable of uniquely identifying the sieve bed assembly;
reading the identification data from the identification device via a reader; and
storing the read identification data in a database.

2. The method of claim 1, wherein the reading is performed at a manufacturing facility.

3. The method of claim 2, wherein the reading is performed by a reader on an oxygen concentrator.

4. The method of claim 3, further comprising sending the read identification data to a server via a transceiver on the oxygen concentrator.

5. The method of claim 4, further comprising correlating the read identification data to manufacturing information relating to the sieve bed assembly.

6. The method of claim 5, further comprising:
collecting operational data from the oxygen concentrator;
transmitting the collected operational data to a database; and
correlating the read identification data to the collected operating information.

7. The method of claim 1, wherein the identification device is one of an RFID tag sending an RFID signal to the reader or an NFC tag sending an NFC signal to the reader.

8. The method of claim 1, wherein the identification device is a label including a graphic of the identification data, and wherein the reader is an optical scanner.

9. The method of claim 1, further comprising:
predicting an end of life of the sieve bed assembly; and
correlating the predicted end of life with the read identification data.

10. The method of claim 1, wherein the sieve bed assembly includes a readable and writable memory device storing the identification data.

11-20. (canceled)

21. A sieve bed assembly comprising:
a canister having an intake;
adsorbent material for producing oxygen enriched air from compressed air in a swing adsorption process, wherein the adsorbent material is within the canister; and
an identification device including identification data for the sieve bed assembly;
wherein the identification data is capable of uniquely identifying the sieve bed assembly; and wherein the identification device is configured to allow a reader to read the identification data from the identification device.

22. The sieve bed assembly of claim 21, wherein the identification device is one of an RFID tag operable to send an RFID signal to the reader or an NFC tag operable to send an NFC signal to the reader.

23. The sieve bed assembly of claim 21, wherein the identification device is an EEPROM.

24. (canceled)

25. An oxygen concentrator comprising:
a retention mechanism for holding a sieve bed assembly having a canister with an adsorbent material and an identification device including identification data uniquely identifying the sieve bed assembly;
a compression system including a compressor coupled to the canister, the compressor configured to compress air for the canister to produce oxygen enriched air in a swing adsorption process;
a reader operable to read the identification data from the identification device;
a transceiver; and
a controller coupled to the reader and the transceiver, the controller operable to cause the reader to read the identification data and send the read identification data to a remote external device via the transceiver.

26. The oxygen concentrator of claim 25, wherein the identification device is one of: an RFID tag operable to send an RFID signal to the reader; and an NFC tag operable to send an NFC signal to the reader.

27. The oxygen concentrator of claim 25, further comprising a printed circuit board including the controller and the reader in fixed relation to the retention mechanism, wherein the controller is operable to determine whether the sieve bed assembly is properly installed in the retention mechanism based on the strength of the signal received by the reader.

28. The oxygen concentrator of claim 25, wherein the remote external device is a cloud based server.

29. The oxygen concentrator of claim 25, wherein the remote external device is a portable computing device.

30. The oxygen concentrator of claim 25, wherein the remote external device is operable to predict an end of life of the sieve bed assembly and correlate the predicted end of life with the read identification data.

31. The oxygen concentrator of claim 25, wherein the identification device is a readable and writable memory device.
